SEQUENCE LISTING (6 sequences)

SEQ ID NO: 1 - DNA, 1161 bp, Homo sapiens
aattcggcacgagccccactctgcctcctgctcccccagggcagcaccatgtggcccctgtggctctgctgggcactctgggtgctgcccctggctggccccggggcggccctgaccgaggagcagctcctgggcagcctgctgcggcagctgcagctcagcgaggtgcccgtactggacagggccgacatggagaagctggtcatccccgcccacgtgagggcccagtatgtagtcctgctgcggcgcagccacggggaccgctcccgcggaaagaggttcagccagagcttccgagaggtggccggcaggttcctggcgtcggaggccagcacacacctgctggtgttcggcatggagcagcggctgccgcccaacagcgagctggtgcaggccgtgctgcggctcttccaggagccggtccccaaggccgcgctgcacaggcacgggcggctgtccccgcgcagcgcccaggcccgggtgaccgtcgagtggctgcgcgtccgcgacgacggctccaaccgcacctccctcatcgactccaggctggtgtccgtccacgagagcggctggaaggccttcgacgtgaccgaggccgtgaacttctggcagcagctgagccggccccggcagccgctgctgctacaggtgtcggtgcagagggagcatctgggcccgctggcgtccggcgcccacaagctggtccgctttgcctcgcagggggcgccagccgggcttggggagccccagctggagctgcacaccctggacctcagggactatggagctcagggcgactgtgaccctgaagcaccaatgaccgagggcacccgctgctgccgccaggagatgtacattgacctgcaggggatgaagtgggccaagaactgggtgctggagcccccgggcttcctggcttacgagtgtgtgggcacctgccagcagcccccggaggccctggccttcaattggccatttctggggccgcgacagtgtatcgcctcggagactgcctcgctgcccatgatcgtcagcatcaaggagggaggcaggaccaggccccaggtggtcagcctgcccaacatgagggtgcagaagtgcagctgtgcctcggatggggcgctcgtgccaaggaggctccagccataggcgcctggtgta

SEQ ID NO: 2 - protein, 366 aa, Homo sapiens
MWPLWLCWALWVLPLAGPGAALTEEQLLGSLLRQLQLSEVPVLDRADMEKLVIPAHVRAQYVVLLRRSHGDRSRGKRFSQSFREVAGRFLASEASTHLLVFGMEQRLPPNSELVQAVLRLFQEPVPKAALHRHGRLSPRSAQARVTVEWLRVRDDGSNRTSLIDSRLVSVHESGWKAFDVTEAVNFWQQLSRPRQPLLLQVSVQREHLGPLASGAHKLVRFASQGAPAGLGEPQLELHTLDLRDYGAQGDCDPEAPMTEGTRCCRQEMYIDLQGMKWAKNWVLEPPGFLAYECVGTCQQPPEALAFNWPFLGPRQCIASETASLPMIVSIKEGGRTRPQVVSLPNMRVQKCSCASDGALVPRRLQP

SEQ ID NO: 3 - DNA, 36 bp, Artificial Sequence
  forward primer
agaattcaagatgtggcccctgtggctctgctgggc

SEQ ID NO: 4 - DNA, 40 bp, Artificial Sequence
  reverse primer
ttctagactatggctggagcctccttggcacgagcgcccc

SEQ ID NO: 5 - DNA, 44 bp, Artificial Sequence
  forward primer
gcgtccgcgacgacggctccgaccgcacctccctcatcgactcc

SEQ ID NO: 6 - DNA, 44 bp, Artificial Sequence
  reverse primer
ggagtcgatgagggaggtgcggtcggagccgtcgtcgcggacgc

(19) United States
(12) Patent Application Publication
(10) Pub. No.: US 2006/0099576 A1
(43) Pub. Date: May 11, 2006

(54) Method for diagnosing a pre-neoplastic or neoplastic lesion in transitional epithelial cells

(76) Inventor: Siamak Tabibzadeh, Albertson, NY (US)

Correspondence Address: Craig J. Arnold;Amster, Rothstein & Ebenstein, 90 Park Avenue, New York, NY, 10016, US

(21) Appl. No.: 10/014,320

(22) Filed: Dec. 11, 2001

Related U.S. Application Data
(60) Provisional application No. 60/255,641, filed on Dec. 14, 2000.

(51) Int. Cl. C12Q 1/68; G01N 33/574

(52) U.S. Cl. 435/6; 435/7.23

(57) ABSTRACT

The present invention provides a method for determining whether a subject has a pre-neoplastic or neoplastic lesion in transitional epithelial cells. The present invention further provides a method for assessing the efficacy of therapy to treat a pre-neoplastic or neoplastic lesion in transitional epithelial cells in a subject who has undergone or is undergoing treatment for a pre-neoplastic or neoplastic lesion in transitional epithelial cells. Finally, the present invention provides a method for assessing the prognosis of a subject who has a pre-neoplastic or neoplastic lesion in transitional epithelial cells.

This application claims the benefit of U.S. Provisional Application No. 60/255,641, filed Dec. 14, 2000.

### STATEMENT OF GOVERNMENT INTEREST

This invention was made with government support under NIH Grant No. CA46866. As such, the United States government has certain rights in this invention.

### BACKGROUND OF THE INVENTION

Transitional-cell carcinoma (TCC) is a special type of cancer which occurs in transitional epithelium. Transitional epithelium lines the entire urinary tract, including the bladder, pelvis of the kidney, ureter, and urethra. In addition, transitional epithelium also lines a region which resides between the anal canal and the rectosigmoid colon. The urinary bladder is the most common site of occurrence of TCC.

Bladder cancer is the fourth most common malignancy diagnosed in men, and the eighth most common malignancy in women (Metts et al., 2000). The male:female incidence of bladder cancer is about 5:2, and it has been estimated that there are approximately 54,500 new cases each year in the United States (Beers and Berkow, 1999).

Bladder cancer arises from superficial epithelium of the bladder. Its prognosis varies from a superficial, well-differentiated disease process that does not significantly impact survival, to extremely malignant tumors which render bleak any prospects for survival (Metts et al., 2000). Staging characterizes the disease as superficial or invasive. Invasive bladder cancers carry a less-favorable prognosis. Approximately 70-80% of patients present with superficial disease, while the remainder progress to, or present with, invasive disease (Farrow, 1992; and Baniel, 1999). In patients with superficial malignancies, death from bladder cancer is very rare. In contrast, in patients with deeply-invasive lesions of the bladder musculature, survival is poor (Beers and Berkow, 1999).

In the United States, nearly all cases (more than 90%) of bladder cancer are transitional-cell carcinomas, which arise from the transitional epithelium of the bladder (Metts et al., 2000; and Soloway, 1992). Squamous-cell carcinomas, which arise in the squamous-cell epithelium of the bladder, are much less prevalent, and are generally associated with parasitic infestation or chronic irritation of the mucosa. Finally, adenocarcinomas, which arise in the glandular epithelium of the bladder, may occur as primary tumors, but are also quite rare (Beers and Berkow, 1999).

Transitional-cell carcinoma of the bladder may range from a superficial, well-differentiated papillary tumor to a highly-invasive, poorly-differentiated tumor at presentation (Beers and Berkow, 1999). Superficial TCC of the bladder is defined as a transitional-cell tumor that is confined to surface epithelium and that has not invaded the lamina propria (Stage Ta), or that has invaded the lamina propria (Stage T1). Noninvasive (superficial) transitional-cell carcinomas of the bladder have two distinct morphologies: papillary transitional-cell carcinomas and carcinoma in situ (CIS). Papillary TCCs are often multifocal, but have limited potential for invasive growth or a lethal outcome. In contrast, flat tumors, or CIS, frequently progress to invasive cancer (Spruck et al., 1994). Patients with invasive cancer, including localized stages (Stages T2 and T3) and more extensive disease (Stages T3 and T4), have poorer prognoses.

The management options for bladder cancer depend on the stage of the disease at presentation. Management of invasive disease warrants extensive surgery, which is the best single-treatment modality. Chemotherapy and radiotherapy are implemented in the management of progression or metastasis. In contrast, most superficial diseases are managed by transurethral resection, cytoscopic follow-up, and additional intravesical therapy when high-risk parameters for recurrence or progression exist (Baniel, 1999).

When treating patients with superficial bladder cancer, the initial tumor is removed. Thereafter, the other parts of the urothelium must be assessed for the presence of pre-malignant or malignant abnormalities. The stage of the tumor (i.e., the depth and extent of the invasion) then must be determined. Furthermore, a determination must be made as to whether or not additional treatment is indicated (e.g., cystectomy, intravesical chemotherapy or immunotherapy, and radiotherapy). Finally, the patient must be monitored for the development of subsequent tumors (Soloway, 1992). Treatment and follow-up depend upon tumor grade and stage (Madrid Garcia et al., 1998), which are the best prognostic indicators (Baniel, 1999).

Bladder cancer presents with various clinical symptoms and signs. Microscopic hematuria may be the earliest sign, although dysuria and pyuria are also common at presentation. Pelvic pain may occur with advanced disease, and a mass may be palpable on bimanual examination.

Numerous tests may be carried out in the diagnosis of bladder cancer. Filling defects of the bladder on cystography or in the cystographic phase of intravenous urography may suggest a bladder tumor. In addition, urinary cytology is useful in screening for possible urinary tract neoplasia in high-risk populations, and in following patients after resection of bladder tumors. Unfortunately, inflammatory or reactive hyperplastic lesions of the urinary tract, and cytotoxic drugs for nonurogenital carcinoma, may produce false-positive results, and false-negative findings are usually associated with neoplasia that appears low-grade on histology. Bimanual examination under anesthesia, and pelvic computer tomography, MRI, and ultrasonography, may assist in determining the stage of the disease. However, bladder cancer is usually diagnosed by cystoscopy and transurethral biopsy or resection (Beers and Berkow, 1999).

Successful management of TCC of the bladder greatly depends upon regular monitoring and early detection of persistent or recurrent carcinomas. As with other cancers, a correlation exists between the tumor burden in a patient with bladder cancer, and that patient's chances of survival. The mortality from bladder cancer can be significantly reduced if tumors are found and treated at an early stage. Many tumors do not produce any clinical signs or symptoms before they reach a considerable size. Despite the various methods for detecting, diagnosing, and treating bladder cancer, the disease remains prevalent in both women and men and, at a progressed stage, can be fatal. Clearly, alternative strategies for detection, including the development of markers that can identify bladder tumors at an early stage and that can distinguish among the various types and stages of bladder cancer, are needed to improve survival in bladder cancer patients.

Currently, there are very few markers available that are specific for the diagnostic characterization of tumors. Carcinomembryonic antigen (CEA), prostatic specific antigen (PSA), and carcinoma-125 (CA-125) are among the most widely used tumor markers. The development of these markers has made it possible to detect tumors at an early stage, and to reduce the morbidity and mortality from cancer. However, the expression of CEA or CA-125 is not specific, and these proteins are expressed in a variety of conditions not related to the cancer. Accordingly, these markers cannot be used for mass screening of the general population: such usage would lead to a high number of false positives, requiring additional testing to rule out the presence of cancer. Thus, to achieve mass screening, there is a need for a specific and sensitive test to detect bladder cancers, particularly at an early stage.

Recently, a new member of the TGF-β superfamily, lefty-1, was recognized for its distinct asymmetric expression in gastrulating mouse embryos (Meno et al., 1996). Lefty-A is the human homologue of lefty-1. Lefty-A is also known as endometrial bleeding associated factor (ebaf) protein, which is associated with abnormal endometrial bleeding (Kothapalli et al., 1997). Ebaf is highly expressed in human endometrium prior to and during menstrual bleeding or abnormal uterine bleeding (Kothapalli et al., 1997). The amino acid sequence of the ebaf protein shows homology with, and structural features of, members of the TGF-β superfamily (Kothapalli et al., 1997), and ebaf is also recognized as a member of the TGF-β superfamily.

The ebaf gene is located on human chromosome 1, at band q42.1, and its nucleotide and deduced amino acid sequences are known. In view of the similarity in the nucleotide sequences of lefty-1 and ebaf, Kosaki et al. (1999) hypothesized that there may be mutations in the ebaf sequence in patients with laterality defects. Kosaki et al. (1999) went on to show that mutations in the ebaf gene are associated with left-right axis malformations in humans. A second human gene, lefty-B, also has been described. In mice, both the lefty-1 gene and the lefty-2 gene reside on chromosome 1H2. In humans, both the lefty-A (ebaf) gene and the lefty-B gene map to human syntenic region 1q42, and are separated from each other by 50 kb. The nucleotide sequences of lefty-A (ebaf) and lefty-B are 97% identical, so these proteins are more closely related to each other than to either of the mouse homologues.

It is known that ebaf mRNA is highly expressed in neoplastic cells that give rise to adenocarcinomas originating from the colon, ovary, pancreas, and testis (Tabibzadeh et al., 1997; U.S. Pat. No. 5,916,751). It is also known that other members of the TGF-β superfamily have been found in human bladder carcinomas (Miyamoto et al., 1995; and Eder et al., 1996). However, prior to the present invention, it was not known that ebaf protein is highly expressed in cells of transitional-cell carcinomas and atypia/dysplasia of transitional epithelium. Moreover, prior to the present invention, it was not known that ebaf is secreted into bodily fluids, particularly the urine.

### SUMMARY OF THE INVENTION

The present invention is based upon the discovery that endometrial bleeding associated factor (ebaf), a secreted protein, is found in the urine of subjects who have transitional-cell carcinoma (TCC) of the bladder. This discovery has broad implications in the diagnosis and treatment of TCCs of the urinary tract, and in the monitoring of TCC therapy.

Accordingly, it is an object of the present invention to provide a method for determining whether a subject has a pre-neoplastic or neoplastic lesion in transitional epithelial cells, by assaying a diagnostic sample of the subject for ebaf expression, wherein ebaf expression elevated above normal is diagnostic of a pre-neoplastic or neoplastic lesion in transitional epithelial cells.

It is also an object of the present invention to provide a method for assessing the efficacy of therapy to treat a pre-neoplastic or neoplastic lesion in transitional epithelial cells in a subject who has undergone or is undergoing treatment for a pre-neoplastic or neoplastic lesion in transitional epithelial cells, by assaying a diagnostic sample of the subject for ebaf expression, wherein normal ebaf expression is indicative of successful therapy to treat a pre-neoplastic or neoplastic lesion in transitional epithelial cells, and ebaf expression elevated above normal is indicative of a need to continue therapy to treat a pre-neoplastic or neoplastic lesion in transitional epithelial cells.

Finally, it is an object of the present invention to provide a method for assessing the prognosis of a subject who has a pre-neoplastic or neoplastic lesion in transitional epithelial cells, by assaying a diagnostic sample of the subject for ebaf expression, wherein the subject's prognosis improves with a decrease in ebaf expression in the diagnostic sample of the subject.

Additional objects of the present invention will be apparent from the description which follows.

### BRIEF DESCRIPTION OF THE FIGURES

FIG. 1 shows that ebaf proteins are secreted. Lanes 1 and 2: ³⁵S methionine-labeled ebaf was immunoprecipitated from the culture medium of human embryonic kidney epithelial (HEK)-293 cells (lane 1) or HEK-293 cells transfected with ebaf (lane 2) using A351 anti-ebaf antibody—the rabbit polyclonal antibody to the carboxy-terminus (C-terminus) of ebaf (amino acid residues 351-367). The immunoprecipitates then were subjected to SDS-PAGE, followed by autoradiography. Lanes 3 and 4: Ebaf in the culture medium of HEK-293 cells (lane 3) or HEK-293 cells transfected with ebaf (lane 4) was immunoaffinity-purified and subjected to SDS-PAGE, followed by Western blotting using the A351 antibody. Numbers at the left of the figure correspond with the molecular weights (in kD) of ebaf in its precursor and processed forms. IP: immunoprecipitate; WB: Western blotting

FIG. 2 depicts detection of ebaf protein by the A351 and A31 antibodies. A31 is the rabbit polyclonal antibody to the amino-terminus (N-terminus) of ebaf (amino acid residues 31-43). Ebaf protein was affinity-purified from the culture media of HEK-293 cells transfected using the A351 (lanes 1 and 3) or the A31 (lanes 2 and 4) antibody. 200 ng of the purified protein were subjected to Western blotting. The blots were probed with A351 (lanes 1 and 2) or A31 (lanes 3 and 4) antibodies. Numbers at the left of the figure correspond with the molecular weights (in kD) of ebaf in its precursor and processed forms.

FIG. 3 illustrates the identification of the endoproteolytic cleavage sites of ebaf. Ebaf shows two potential convertase cleavage sites: RGKR (amino acid residues 74-77) and RHGR (amino acid residues 132-135). HEK-293 cells were transfected with wild-type or mutated GGKG (amino acid residues 74-77) and GHGR (amino acid residues 132-135) forms of ebaf. The effect of these mutations on ebaf processing was analyzed by Western blotting using anti-ebaf antibody, A351. Numbers at the left of the figure correspond with the molecular weights (in kD) of ebaf in its precursor and processed forms.

FIG. 4 illustrates that ebaf is a glycosylated protein. A: The conditioned medium of HEK-293 cells stably transfected with ebaf sense cDNA (lanes 1 and 3) and HEK-293 cells transiently transfected with ebaf sense cDNA from several clones mutated at the potential glycosylation site (lanes 2 and 4) were subjected to Western blotting. The blot was stained with the A351 polyclonal antibody. Molecular weights are shown in kD. B: The conditioned medium of HEK-293 cells stably transfected with ebaf sense cDNA was incubated without (lane 1) and with Endo H (lane 2) and with PNGase (lane 3), as described in the text, and then was subjected to Western blotting. The blot was stained with the A351 antibody. Molecular weights are shown in kD.

FIG. 5 shows the presence of ebaf protein in bladder carcinomas. 20 μg of tissue lysate from a normal bladder mucosa (lane 1) and bladder carcinomas (lanes 2-8) were subjected to Western blotting. The blot was probed with A351 anti-ebaf antibody. Numbers at the left of the figure correspond with the molecular weights (in kD) of ebaf in its precursor and processed forms.

FIG. 6 also shows the presence of ebaf protein in bladder carcinomas. A: 20 ng of purified ebaf (lane 1), and 20 μg of protein from tissue lysates from bladder carcinomas (lanes 2, 4, and 6) and from adjacent normal bladder mucosa (lanes 3, 5, and 7), were subjected to Western blotting. The blots were probed with A351 anti-ebaf antibody. Molecular weights are shown in kD. B: Sections of a urothelial mucosa lined with atypical epithelium (panels A and C) or adjacent papillary transitional-cell carcinoma (panels B and D) were immunostained for ebaf using A351 antibody. Arrows point to some of the strongly stained cells. The dotted lines in panels A and C show the boundaries of the surface epithelium. The dashed lines in panels B and D show the boundaries of the papillary carcinomas. SE: surface epithelium; LP: lamina propria; C: core of a papillary structure

FIG. 7 shows ebaf present in urine of either normal subjects or patients with hyperplasia, atypia, and carcinoma of transitional epithelium. All molecular weights are in kD. FIG. 7A shows the presence of ebaf in the urine of normal subjects. Purified ebaf (lane 1), and ebaf purified from 2.5 ml of urine samples of normal subjects (lanes 2-25), were subjected to Western blotting. The blots were probed with A351 antibody. FIG. 7B shows the presence of ebaf in the urine of patients without and with atypia. Ebaf purified from 2.5 ml of urine samples taken from patients with chronic inflammation (lane 1), hyperplasia (lane 2), or atypia (lanes 4-7) was subjected to Western blotting. The blots were probed with A351 antibody. FIG. 7C shows the presence of ebaf in the urine of patients with transitional-cell carcinoma (TCC). Ebaf purified from 2.5 ml of urine samples taken from patients with low-grade papillary transitional-cell carcinomas (lanes 1-11), carcinoma in situ (lanes 12-13), or high-grade papillary transitional-cell carcinoma (lanes 14-17) was subjected to Western blotting. The blots were probed with A351 antibody. FIG. 7D shows the presence of ebaf in the urine of normal subjects and bladder cancer patients. Ebaf purified from urine (lanes 1-3) and concentrated urine (lanes 4-6) was subjected to Western blotting. The blots were probed with A351 antibody. Lanes 1 and 6 show ebaf in the urine from a normal subject. Lanes 2, 3, 4, 5 show ebaf in the urine from patients with papillary TCC.

FIG. 8 depicts ebaf protein in the sera of normal subjects and in the sera of patients with bladder carcinomas. All molecular weights are in kD. A: Purified ebaf (lane 1) and ebaf affinity-purified from sera of normal patients (lanes 2-3: male; lane 4: female, day 3 of the menstrual cycle; lane 5: female, day 3 after ovulation; lane 6: female, day 10 after ovulation; lane 7: female, first day of menses) and bladder cancer patients (lanes 8-14) were subjected to Western blotting. The blots were probed with A351 anti-ebaf antibody. B: Purified ebaf (lane 1) and ebaf affinity-purified from urine (upper panel) and sera (lower panel) of control patient without TCC (lane 2) and with TCC (lanes 3-9) were subjected to Western blotting. The blots were probed with A351 anti-ebaf antibody.

FIG. 9 depicts the nucleotide sequence and corresponding amino acid sequence for ebaf.

### DETAILED DESCRIPTION OF THE INVENTION

The present invention provides a method for determining whether a subject has a pre-neoplastic or neoplastic lesion in transitional epithelial cells. As used herein, the “subject” is a mammal, including, without limitation, a cow, dog, human, monkey, mouse, pig, or rat, but is preferably a human. The method of the present invention comprises assaying a diagnostic sample of the subject for expression of endometrial bleeding associated factor (ebaf), wherein ebaf expression elevated above normal is diagnostic of a pre-neoplastic or neoplastic lesion in transitional epithelial cells.

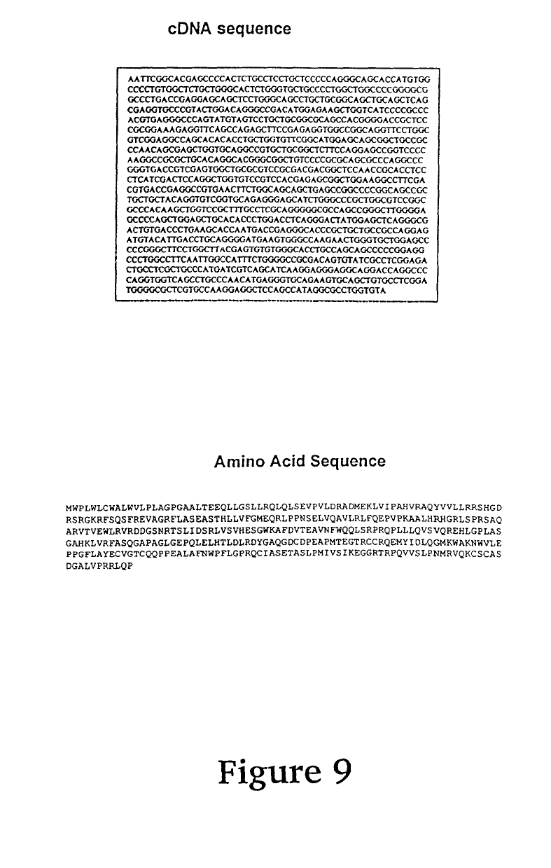

Unless otherwise indicated, “ebaf” includes both an ebaf (lefty-A) protein and an “ebaf analogue”. As used herein, “ebaf protein” includes the 42-kD pro-protein (precursor) form of ebaf, as disclosed herein, as well as the 34- and 28-kD C-terminus polypeptides processed from the precursor by proteolytic cleavage and any other forms of the precursor produced by post-translational modification (e.g., glycosylation, etc.). Ebaf protein has the amino acid sequence set forth in FIG. 9, or a portion thereof corresponding to the 34- or 28-kD processed forms of the protein. An “ebaf analogue”, as used herein, is a variant of the ebaf protein that has 60% or greater (preferably, 70% or greater) amino-acid-sequence homology with the ebaf protein, as well as a variant of the ebaf protein that has a homologous three-dimensional conformation. Additionally, the term “ebaf analogue”, as defined herein, includes peptides related to ebaf, particularly lefty-B, lefty-1, lefty-2, and other proteins that have an amino acid sequence similar to that of ebaf. Preferably, the ebaf analogue is lefty-B. Ebaf and ebaf analogues may be produced synthetically or recombinantly, or may be isolated from native cells. Ebaf is preferably produced recombinantly, using conventional techniques and cDNA encoding ebaf (FIG. 9).

The method of the present invention may be used to determine whether a subject has a pre-neoplastic or neoplastic lesion in transitional epithelial cells, thereby permitting the diagnosis of such a lesion in the subject. As used herein, “a pre-neoplastic or neoplastic lesion in transitional epithelial cells” includes, without limitation, morphological irregularities in transitional epithelial cells of transitional epithelium, as well as pathologic proliferation of transitional epithelial cells of transitional epithelium, as compared with normal proliferation in the same type of tissue. Transitional epithelium is a specialized type of epithelium that covers the organs of the urinary tract (e.g., the bladder, pelvis of the kidney, ureter, and urethra). It also may be found in other parts of the body (e.g., the transitional zone between the anal canal and the colonic mucosa). Transitional epithelium may be distinguished from both squamous epithelium (which may give rise to squamous-cell carcinoma) and glandular epithelium (which may give rise to adenocarcinoma).

As used herein, “neoplastic lesion”, or neoplasia, refers to the uncontrolled and progressive multiplication of tumor cells in transitional epithelium, under conditions that would not elicit, or would cause cessation of, multiplication of normal transitional epithelial cells. Neoplasia results in the formation of a “neoplasm”, which is defined herein to mean any new and abnormal growth, particularly a new growth of tissue, in which the growth of cells is uncontrolled and progressive. Neoplasms include benign tumors and malignant tumors (e.g., TCCs of the urinary tract, including carcinoma in situ and papillary transitional-cell carcinoma) that are either invasive or noninvasive. Malignant neoplasms are distinguished from benign in that the former show a greater degree of anaplasia, or loss of differentiation and orientation of cells, and have the properties of invasion and metastasis. Thus, neoplasia includes “cancer”, which herein refers to a proliferation of tumor cells in transitional epithelium having the unique trait of loss of normal controls, resulting in unregulated growth, lack of differentiation, local tissue invasion, and/or metastasis. In one embodiment of the present invention, the neoplastic lesion in transitional epithelial cells is a transitional-cell carcinoma of the urinary tract, such as carcinoma in situ and papillary transitional-cell carcinoma.

As used herein, “pre-neoplastic lesion” refers to a lesion in transitional epithelial cells that has the biologic potential to become neoplastic. Examples of pre-neoplastic lesions in transitional epithelial cells include, without limitation, atypia and dysplasia. As used herein, “atypia” refers to irregular or nonconforming transitional epithelial cells. As further used herein, “dysplasia” refers to the abnormal development of transitional epithelial cells, particularly the pathologic alteration in size, shape, and organization of adult transitional epithelial cells (e.g., pre-malignant urothelial atypia/dysplasia). In one embodiment of the present invention, the pre-neoplastic lesion in transitional epithelial cells is pre-malignant urothelial atypia/dysplasia.

According to the method of the present invention, the diagnostic sample of a subject may be assayed in vitro or in vivo. In accordance with the present invention, where the assay is performed in vitro, a diagnostic sample from the subject may be removed using standard procedures. The diagnostic sample may be tissue, particularly any transitional epithelium (including urothelial mucosa from any organ of the urinary tract and a tumor in any transitional epithelium), which may be removed by standard biopsy. In one embodiment of the present invention, the tissue is obtained from the bladder, pelvis of the kidney, ureter, or urethra of the subject. In addition, the diagnostic sample may be a bodily fluid, including cerebrospinal fluid, pericardial fluid, peritoneal fluid, saliva, serum, and urine. Furthermore, the diagnostic sample may be a cytological preparation. Where the diagnostic sample is a cytological preparation, cells (including cancer cells) sloughed off from the lining of the bladder, for example, and subsequently voided in the urine, may be examined in an assay for expression of ebaf. Such a cytological preparation is commonly used in current methods for diagnosing bladder cancer based on cell morphology.

Protein may be isolated and purified from the diagnostic sample of the present invention using standard methods known in the art, including, without limitation, extraction from a tissue (e.g., with a detergent that solubilizes the protein) where necessary, followed by affinity purification on a column, chromatography (e.g., FTLC and HPLC), immunoprecipitation (with an antibody to ebaf, such as A351 antibody, as described herein), and precipitation (e.g., with isopropanol and a reagent such as Trizol). Isolation and purification of the protein may be followed by electrophoresis (e.g., on a SDS-polyacrylamide gel). Nucleic acid may be isolated from a diagnostic sample using standard techniques known to one of skill in the art.

In accordance with the method of the present invention, a pre-neoplastic or neoplastic lesion in transitional epithelial cells in a subject may be diagnosed by assaying a diagnostic sample of the subject for expression of ebaf, wherein ebaf expression elevated above normal is diagnostic of a pre-neoplastic or neoplastic lesion in transitional epithelial cells. As used herein, “expression” means the transcription of the ebaf gene into at least one mRNA transcript, or the translation of at least one mRNA into an ebaf protein, as defined above (i.e., the 42-kD ebaf precursor and any processed forms of ebaf resulting from post-translational modification, including the 34- and 28-kD forms resulting from cleavage of the precursor, as well as other forms produced by glycosylation and other types of modification). Accordingly, a diagnostic sample may be assayed for ebaf expression by assaying for ebaf protein (as defined above), cDNA, or mRNA. The appropriate form of ebaf will be apparent based on the particular techniques discussed herein. Furthermore, as described above, it is contemplated that the diagnostic sample may be assayed for expression of any or all forms of ebaf protein (including precursor, endoproteolytically-processed forms, and other forms resulting from post-translational modification) in order to determine whether a subject or patient has a pre-neoplastic or neoplastic lesion in transitional epithelial cells.

As used herein, “ebaf expression elevated above normal” means expression of ebaf at a level that is significantly greater than the level expected for the same type of diagnostic sample taken from a nondiseased subject or patient (i.e., one who does not have a pre-neoplastic or neoplastic lesion in transitional epithelial cells) of the same gender and of similar age. As further used herein, “significantly greater” means that the difference between the level of ebaf expression that is elevated above normal, and the expected (normal) level of ebaf, is of statistical significance. Preferably, ebaf expression elevated above normal is expression of ebaf at a level that is at least 10% greater than the level of ebaf expression otherwise expected. Where ebaf expression is expected to be absent from a particular diagnostic sample taken from a particular subject or patient, the normal level of ebaf expression for that subject or patient is nil. Where a particular diagnostic sample taken from a particular subject or patient is expected to have a low level of constitutive ebaf expression, that low level is the normal level of ebaf expression for that subject or patient. As disclosed herein, ebaf expression is generally absent, or present at very low levels, in the bladder tissue, serum, and urine of nondiseased control subjects.

Expected or normal levels of ebaf expression for a particular diagnostic sample taken from a subject or patient may be easily determined by assaying nondiseased subjects of a similar age and of the same gender. For example, diagnostic samples may be obtained from at least 30 normal, healthy men between the ages of 25 and 80, to determine the normal quantity of ebaf expression in males. However, to determine the normal quantity of ebaf expression in women, there is a need to collect samples at various stages of the female menstrual cycle, since ebaf is expressed during the late secretory and menstrual phases. Accordingly, diagnostic samples could be collected, for example, from a minimum of five women in each phase of the menstrual cycle—early proliferative, mid-proliferative, late proliferative, early secretory, mid-secretory, late secretory, and menstrual phases—for a total of 35 subjects.

Once the above-described samples have been obtained, the normal quantities of ebaf expression in men and women may be determined using a standard assay for quantification, such as flow cytometry, Western blot analysis, or an ELISA for measuring protein quantities, as described below. For example, an ELISA may be run on each sample in duplicate, and the means and standard deviations of the quantity of the ebaf protein may be determined. If necessary, additional subjects may be recruited before the normal quantities of ebaf expression are quantified.

In accordance with the method of the present invention, a diagnostic sample of a subject may be assayed for ebaf expression using detection methods and assays readily determined from the known art, including, without limitation, immunological techniques, hybridization analysis, fluorescence imaging techniques, and/or radiation detection. For example, according to the method of the present invention, a diagnostic sample of the subject may be assayed for ebaf expression using an agent reactive with ebaf. As used herein, “reactive” means the agent has affinity for, binds to, or is directed against ebaf. As further used herein, an “agent” shall include a protein, polypeptide, peptide, nucleic acid (including DNA or RNA), antibody, Fab fragment, F(ab′)₂ fragment, molecule, compound, antibiotic, drug, and any combinations thereof. A Fab fragment is a univalent antigen-binding fragment of an antibody, which is produced by papain digestion. A F(ab′)₂ fragment is a divalent antigen-binding fragment of an antibody, which is produced by pepsin digestion. Preferably, the agent of the present invention is labeled with a detectable marker. The detection of ebaf expression in the method of the present invention then may be followed by an assay to measure or quantify the extent of ebaf expression in a diagnostic sample of a subject. Such assays are well known to one of skill in the art, and may include flow cytometry, mass spectroscopy, Western blot analysis, or an ELISA for measuring amounts of ebaf protein.

In one embodiment of the present invention, the agent reactive with ebaf is an antibody. As used herein, the antibody of the present invention may be polyclonal or monoclonal, and may be raised against any or all forms of ebaf protein (e.g., the 42-, 34-, or 28-kD protein). In addition, the antibody of the present invention may be produced by techniques well known to those skilled in the art. Polyclonal antibody, for example, may be produced by immunizing a mouse, rabbit, or rat with purified ebaf. Monoclonal antibody then may be produced by removing the spleen from the immunized mouse, and fusing the spleen cells with myeloma cells to form a hybridoma which, when grown in culture, will produce a monoclonal antibody. As disclosed herein, polyclonal anti-ebaf antibodies, A351 and A31, have been produced by immunizing a rabbit.

The antibodies used herein may be labeled with a detectable marker. Labeling of the antibody may be accomplished using one of the variety of labeling techniques, including peroxidase, chemiluminescent labels known in the art, and radioactive labels known in the art. The detectable marker of the present invention may be, for example, a nonradioactive or fluorescent marker, such as biotin, fluorescein (FITC), acridine, cholesterol, or carboxy-X-rhodamine, which can be detected using fluorescence and other imaging techniques readily known in the art. Alternatively, the detectable marker may be a radioactive marker, including, for example, a radioisotope. The radioisotope may be any isotope that emits detectable radiation, such as ³⁵S, ³²P, or ³H. Radioactivity emitted by the radioisotope can be detected by techniques well known in the art. For example, gamma emission from the radioisotope may be detected using gamma imaging techniques, particularly scintigraphic imaging. Preferably, the agent of the present invention is a high-affinity antibody labeled with a detectable marker.

Where the agent of the present invention is an antibody reactive with ebaf, a diagnostic sample (e.g., urine) taken from the subject may be purified by passage through an affinity column which contains ebaf antibody as a ligand attached to a solid support, such as an insoluble organic polymer in the form of a bead, gel, or plate. The antibody attached to the solid support is used in the form of a column. Examples of suitable solid supports include, without limitation, agarose, cellulose, dextran, polyacrylamide, polystyrene, sepharose, or other insoluble organic polymers. The ebaf antibody may be further attached to the solid support through a spacer molecule, if desired. Appropriate binding conditions (e.g., temperature, pH, and salt concentration) may be readily determined by the skilled artisan. In a preferred embodiment, the ebaf antibody is attached to a sepharose column, such as Sepharose 4B. Alternatively, the diagnostic sample of the present invention may be concentrated in a concentration device, such as a Centricon-30 (Amicon Co., Beverly, Mass.), or other similar device. The molecular weight cutoff of the device may be 10,000 kD, for example.

Where the agent is an antibody, a diagnostic sample of the subject may be assayed for ebaf expression using binding studies that utilize one or more antibodies immunoreactive with ebaf, along with standard immunological detection techniques. For example, the ebaf protein eluted from the affinity column, or the retentant in the concentrating device, may be subjected to an ELISA assay, Western blot analysis, flow cytometry, or any other immunostaining method employing an antigen-antibody interaction. Preferably, the diagnostic sample is assayed for ebaf expression using Western blotting.

Alternatively, a diagnostic sample of a subject may be assayed for ebaf expression using hybridization analysis of nucleic acid extracted from the diagnostic sample taken from the subject. According to this method of the present invention, the hybridization analysis may be conducted using Northern blot analysis of mRNA. This method also may be conducted by performing a Southern blot analysis of DNA using one or more nucleic acid probes which hybridize to nucleic acid encoding ebaf. The nucleic acid probes may be prepared by a variety of techniques known to those skilled in the art, including, without limitation, the following: restriction enzyme digestion of ebaf nucleic acid; and automated synthesis of oligonucleotides having sequences which correspond to selected portions of the nucleotide sequence of the ebaf nucleic acid, using commercially-available oligonucleotide synthesizers, such as the Applied Biosystems Model 392 DNA/RNA synthesizer.

The nucleic acid probes used in the present invention may be DNA or RNA, and may vary in length from about 8 nucleotides to the entire length of the ebaf nucleic acid. The ebaf nucleic acid used in the probes may be derived from mammalian ebaf. The nucleotide sequences for both mouse lefty-1 and human lefty-A (ebaf) are known (Meno et al., 1996; and Kothapalli et al., 1997). Using these sequences as probes, the skilled artisan could readily clone corresponding ebaf cDNA from other species. In addition, the nucleic acid probes of the present invention may be labeled with one or more detectable markers. Labeling of the nucleic acid probes may be accomplished using one of a number of methods known in the art—e.g., nick translation, end labeling, fill-in end labeling, polynucleotide kinase exchange reaction, random priming, or SP6 polymerase (for riboprobe preparation)—along with one of a variety of labels—e.g., radioactive labels, such as ³⁵S, ³²P, or ³H, or nonradioactive labels, such as biotin, fluorescein (FITC), acridine, cholesterol, or carboxy-X-rhodamine (ROX). Combinations of two or more nucleic acid probes (or primers), corresponding to different or overlapping regions of the ebaf nucleic acid, also may be used to assay a diagnostic sample for ebaf expression, using, for example, PCR or RT-PCR.

It is contemplated that the diagnostic sample in the present invention frequently will be assayed for ebaf expression not by the subject or patient, nor by his/her consulting physician, but by a laboratory technician or other clinician. Accordingly, the method of the present invention further comprises providing to a subject's or patient's consulting physician a report of the results obtained upon assaying a diagnostic sample of the subject or patient for ebaf expression.

It is also within the confines of the present invention to use detected levels of ebaf expression in an assayed diagnostic sample as a clinical or pathologic staging tool. For example, as disclosed herein, ebaf levels detected in most diagnostic samples taken from patients who had low-grade transitional-cell carcinoma (TCC) of the bladder were lower than those detected in most diagnostic samples taken from patients with high-grade lesions, such as flat carcinoma in situ and high-grade papillary TCC of the bladder. Accordingly, detected levels of ebaf expression in an assayed diagnostic sample may be used to determine the grade or stage of the various tumors and lesions found in transitional epithelium of the bladder and other organs of the urinary tract. In addition, detected levels of ebaf expression in an assayed diagnostic sample may be used to determine whether any treatment method is appropriate for a particular subject or patient who has a pre-neoplastic or neoplastic lesion in transitional epithelial cells.

Furthermore, it is within the confines of the present invention that detected levels of expression of each of the various forms of ebaf (e.g., the 42-, 34-, or 28-kD protein) in an assayed diagnostic sample of a subject or patient may be used to diagnose the particular type of transitional-cell carcinoma in the subject or patient, based upon their relative abundances. As disclosed herein, ebaf precursor and its endoproteolytically-processed forms may be detected in bladder carcinomas to differing extents, depending upon the type of bladder cancer in question. Accordingly, a determination of the extent to which each form of ebaf is expressed in a diagnostic sample of a subject or patient may provide an indication of the type of TCC found in the subject or patient.

The present invention further provides a method for assessing the efficacy of therapy to treat a pre-neoplastic or neoplastic lesion in transitional epithelial cells in a subject or patient who has undergone or is undergoing treatment for a pre-neoplastic or neoplastic lesion in transitional epithelial cells. The method of the present invention comprises assaying a diagnostic sample of the subject or patient for ebaf expression, wherein normal ebaf expression is indicative of successful therapy to treat the pre-neoplastic or neoplastic lesion in transitional epithelial cells, and ebaf expression elevated above normal is indicative of a need to continue therapy to treat the pre-neoplastic or neoplastic lesion in transitional epithelial cells. The pre-neoplastic or neoplastic lesion may be any of those described above, including TCC. The TCC may be a transitional-cell carcinoma of the urinary tract, including TCC of the bladder, pelvis of the kidney, ureter, or urethra (e.g., carcinoma in situ, papillary TCC, etc.), or a TCC of other tissues or organs, outside of the urinary tract, that are lined with transitional epithelium (e.g., the transitional zone between the anal canal and the colonic mucosa). The diagnostic sample may be tissue, a bodily fluid, or a cytological preparation, as described above. The diagnostic sample may be assayed for expression of ebaf in vitro or in vivo. In addition, the diagnostic sample may be assayed for expression of ebaf using all of the various assays and methods of detection and quantification described above. This method of the present invention provides a means of monitoring the effectiveness of therapy to treat a pre-neoplastic or neoplastic lesion in transitional epithelial cells by permitting the periodic assessment of levels of ebaf expression in a diagnostic sample taken from a subject or patient.

According to the method of the present invention, a diagnostic sample of a subject or patient may be assayed, and levels of ebaf expression may be assessed, at any time following the initiation of therapy to treat a pre-neoplastic or neoplastic lesion in transitional epithelial cells. For example, levels of ebaf expression may be assessed while the subject or patient is still undergoing treatment for a pre-neoplastic or neoplastic lesion in transitional epithelial cells. Where levels of ebaf expression detected in an assayed diagnostic sample of the subject or patient continue to remain elevated above normal, a physician may choose to continue with the subject's or patient's treatment for the pre-neoplastic or neoplastic lesion in transitional epithelial cells. Where levels of ebaf expression in an assayed diagnostic sample of the subject or patient decrease through successive assessments, it may be an indication that the treatment for a pre-neoplastic or neoplastic lesion in transitional epithelial cells is working, and that treatment doses could be decreased or even ceased. Where levels of ebaf in an assayed diagnostic sample of the subject or patient do not rapidly decrease through successive assessments, it may be an indication that the treatment for a pre-neoplastic or neoplastic lesion in transitional epithelial cells is not working, and that treatment doses could be increased. Where ebaf expression is no longer detected in an assayed diagnostic sample of a subject or patient at levels elevated above normal, a physician may conclude that the treatment for a pre-neoplastic or neoplastic lesion in transitional epithelial cells has been successful, and that such treatment may cease.

It is also within the confines of the present invention to assess levels of ebaf expression following completion of the subject's or patient's treatment for a pre-neoplastic or neoplastic lesion in transitional epithelial cells, in order to determine whether the pre-neoplastic or neoplastic lesion in transitional epithelial cells has recurred in the subject or patient. Accordingly, an assessment of levels of ebaf expression in an assayed diagnostic sample may provide a convenient way to conduct follow-ups of patients with a pre-neoplastic or neoplastic lesion in transitional epithelial cells. Furthermore, as described above, it is within the confines of the present invention to use assessed levels of ebaf expression in an assayed diagnostic sample as a clinical or pathologic staging tool, as a means of determining the extent of the pre-neoplastic or neoplastic lesion in transitional epithelial cells in the subject or patient, and as a means of ascertaining appropriate treatment options.

It is contemplated that the diagnostic sample of the present invention frequently will be assayed for ebaf expression not by the subject or patient, nor by his/her consulting physician, but by a laboratory technician or other clinician. Accordingly, this method of the present invention further comprises providing to a subject's or patient's consulting physician a report of the results obtained upon assaying a diagnostic sample of the subject or patient for ebaf expression.

A correlation exists, in general, between tumor burden and the survival of a patient who has cancer. The mortality from cancer can be significantly reduced if tumors are found and treated at an early stage. As described below, ebaf expression is detected at low levels in the urine of patients with hyperplasia of the bladder transitional epithelium. Significantly higher levels of ebaf expression, however, are found in the urine of patients with flat carcinoma in situ in the bladder. Moreover, in the majority of patients who had low-grade papillary transitional-cell carcinoma of the bladder, amounts of ebaf detected in the urine were lower than those found in the urine of patients with high-grade carcinomas. Accordingly, the overexpression of ebaf correlates with the degree of morphologic differentiation of transitional-cell carcinomas, with those having a poor prognosis showing the potential to secrete more ebaf into the urine.

In view of the foregoing, it is also contemplated in the present invention that assaying a diagnostic sample for ebaf expression may be a useful tool for providing information concerning the prognosis of a subject or patient who has a pre-neoplastic or neoplastic lesion in transitional epithelial cells. Accordingly, the present invention further provides a method for assessing the prognosis of a subject who has a pre-neoplastic or neoplastic lesion in transitional epithelial cells, comprising assaying a diagnostic sample of the subject for ebaf expression, wherein the subject's prognosis improves with a decrease in ebaf expression in the diagnostic sample of the subject.

In accordance with the method of the present invention, the pre-neoplastic or neoplastic lesion in transitional epithelial cells may be any of those described above, including TCC. The TCC may be a transitional-cell carcinoma of the urinary tract, including TCC of the bladder, pelvis of the kidney, ureter, or urethra (e.g., carcinoma in situ, papillary TCC, etc.), or a TCC of other tissues or organs, outside of the urinary tract, that are lined with transitional epithelium (e.g., the transitional zone between the anal canal and the colonic mucosa). The diagnostic sample may be tissue, a bodily fluid, or a cytological preparation, as described above. The diagnostic sample may be assayed in vitro or in vivo. In addition, the diagnostic sample may be assayed using all of the various assays and detection and quantification methods described above. This method of the present invention provides a means of determining the prognosis of a subject or patient diagnosed with a pre-neoplastic or neoplastic lesion in transitional epithelial cells based upon the level of ebaf expression in an assayed diagnostic sample of the subject or patient.

According to the method of the present invention, a diagnostic sample of a subject or patient may be assayed, and levels of ebaf expression may be assessed, at any time following the diagnosis of a pre-neoplastic or neoplastic lesion in transitional epithelial cells in the subject or patient. For example, levels of ebaf expression in an assayed diagnostic sample may be assessed before the subject or patient undergoes treatment for a pre-neoplastic or neoplastic lesion in transitional epithelial cells, in order to determine the subject's or patient's initial prognosis. Additionally, levels of ebaf expression in an assayed diagnostic sample may be assessed while the subject or patient is undergoing treatment for a pre-neoplastic or neoplastic lesion in transitional epithelial cells, in order to determine whether the subject's or patient's prognosis has become more or less favorable.

Where levels of ebaf expression detected in an assayed diagnostic sample of the subject or patient continue to remain significantly elevated above normal, a physician may conclude that the subject's or patient's prognosis is poor. Where levels of ebaf expression in an assayed diagnostic sample of the subject or patient decrease through successive assessments, it may be an indication that the subject's or patient's prognosis is improving. Where levels of ebaf in an assayed diagnostic sample of the subject or patient do not decrease significantly through successive assessments, it may be an indication that the subject's or patient's prognosis is not improving. Where ebaf expression is no longer detected in an assayed diagnostic sample of the subject or patient at levels significantly elevated above normal, a physician may conclude that the subject's or patient's prognosis is favorable.

It is contemplated that the diagnostic sample of the present invention frequently will be assayed for ebaf expression not by the subject or patient, nor by his/her consulting physician, but by a laboratory technician or other clinician. Accordingly, the method of the present invention further comprises providing to a subject's or patient's consulting physician a report of the results obtained upon assaying a diagnostic sample of the subject or patient for ebaf expression.

The discovery that ebaf can be detected at above-normal levels in the bodily fluids and tissues of subjects suffering from TCC or pre-neoplastic lesions in transitional epithelial cells provides a means for identifying patients with TCC or pre-neoplastic lesions in transitional epithelial cells, and presents the potential for commercial application in the form of a noninvasive urine or serum test (or a test involving another bodily fluid) for the diagnosis of pre-neoplastic lesions in transitional epithelial cells, and TCC of the urinary tract and of other tissues and organs that are lined with transitional epithelium. The development of such a test could provide general mass-screening procedures. Such procedures may be used for mass screening of the general population, and for screening subjects or patients who have no symptoms or signs of disease. General mass-screening procedures can assist in the early detection and diagnosis of such cancers, and can provide a method for the follow-up of patients in whom above-normal ebaf expression has been detected. Accordingly, the present invention further provides a kit for use as an assay of pre-neoplastic and neoplastic lesions in transitional epithelial cells, comprising an agent reactive with ebaf. The agent may be any of those described above, and may be used in any of the above-described assays or methods for detecting or quantifying ebaf expression. In addition, the kit may detect any or all forms of ebaf (e.g., the 42-, 34-, or 28-kD protein) or fragments thereof.

The present invention is described in the following Experimental Details section, which is set forth to aid in the understanding of the invention, and should not be construed to limit in any way the scope of the invention as defined in the claims which follow thereafter.

### Experimental Details

1. Introduction

Ebaf (lefty-A) is a member of the family of morphogens that direct the left-right (L-R) asymmetry of the vertebrate body plan from an originally symmetric embryo (Meno et al., 1996, 1997, 1998; and Kothapalli et al., 1997). Consistent with a role in the development of laterality, Kosaki et al. (1999) reported that ebaf, the presumed human homologue of mouse lefty-1, carries mutations in individuals who show abnormal axis development. However, there are clear indications that this embryonic signal continues, during adulthood, to regulate multiple functions in a variety of organs.

Ebaf was originally cloned as a human homologue of lefty-1 for its role in normal and abnormal uterine bleeding. For this reason, it is also known as endometrial bleeding associated factor (ebaf). More recently, it was discovered that lefty-1 transcript and translation products are induced during the course of osteoblastic cell differentiation. Lefty-1 modulated the induction of alkaline phosphatase (ALP) by these cells (Seth et al., 2000). These findings show that the ebaf gene does not become silent after embryonic development, and is likely to continue to function throughout life. Besides these pleiotropic actions in homeostasis of normal tissues, ebaf is considered to play a part in the pathogenesis of a distinct group of tumors. Specifically, it is known that ebaf mRNA is highly expressed in the neoplastic cells that give rise to adenocarcinomas originating from colon, ovary, pancreas, and testis (Tabibzadeh et al., 1997; U.S. Pat. No. 5,916,751). In contrast, ebaf is not expressed in nonepithelial tumors, such as melanoma, sarcoma, and lymphoma. Prior to the present invention, there was no evidence that ebaf protein is synthesized, processed, and released by the cells of transitional-cell carcinomas or atypia/dysplasia of transitional epithelium. Moreover, prior to the present invention, there was no evidence that ebaf is secreted into bodily fluids, particularly the urine.

TGF-β superfamily members are all processed before activation, suggesting that protein cleavage is an essential step for ebaf activation and, consequently, for cell signaling. Like many other proteins, members of the TGF-β superfamily are synthesized as large, inactive precursor proteins, that must be proteolytically processed to release the bioactive polypeptides (Sha et al., 1989). Among the members of the TGF-β super-family, processing of the TGF-β1 precursor has been widely studied. Based on the findings, it is clear that proteolytic cleavage by a convertase, furin, is an essential step in the formation of the biologically-active TGF-β1 polypeptide.

TGF-β1 is expressed as an inactive 55-kD precursor protein that, after enzymatic cleavage, produces a 12.5-kD carboxy-terminus polypeptide that, once dimerized, forms a 25-kD biologically-active homodimer (Dubois et al., 1995). However, despite its biological importance, virtually nothing is known about the endoproteolytic cleavage of ebaf protein, and the biological activity of its released form. Accordingly, the inventor has analyzed herein the secretion and cleavage of ebaf, and have identified the cleavage sites of the ebaf protein. Because bladder cancer remains a common cancer in both women and men (Metts et al., 2000), the inventor used transitional-cell carcinoma (TCC) of the bladder as a paradigm to assess synthesis and release of ebaf. To determine that the cleavage of ebaf in bladder carcinomas releases biologically-active products, the transforming ability of ebaf purified from urine of bladder cancer patients was compared to the activity of a recombinant ebaf protein.

2. Materials and Methods

The full length, 1.961-kb ebaf (lefty-A) cDNA was derived from a human placental cDNA library (Lamb et al., 1993). A 1.1-kb cDNA fragment of glyceraldehyde 3-phosphate dehydrogenase (GAPDH) was obtained from Clontech (Palo Alto, Calif.). Other materials included deoxycytidine 5′-triphosphate dCTP α-³²P (3000 Ci/mmol) (Dupont NEN Research Products, Boston, Mass.), Prime-a-Gene labeling kit (Promega, Madison, Wis.), Nick column (Pharmacia Biotech, Piscataway, N.J.), Hybond nylon membrane and enhanced chemiluminescence system (Amersham, Arlington Heights, Ill.), Kodak-OMAT films (Sigma Chemical Company, St. Louis, Mo.), Coomassie Plus Protein Assay Reagent (Pierce, Rockford, Ill.), nitrocellulose membrane (MSI, Westborough, Mass.), biotin-labeled goat anti-rabbit antiserum and avidin-biotin complex (ABC) reagent (Vector Laboratories, Burlingame, Calif.), Protein G Plus Agarose (Santa Cruz Biotechnology, Santa Cruz, Calif.), enhanced chemiluminescence system (Boehringer Mannheim, Indianapolis, Ind.), PVDF membranes (Bio-Rad Laboratories, Hercules, Calif.), and Kodak-OMAT films (Sigma Chemical Company, St. Louis, Mo.). Chinese hamster ovary (CHO) and human embryonic kidney epithelial (HEK)-293 cells were obtained from American Type Culture Collection (Manassas, Va.). All other chemicals were obtained from either Sigma Chemical Company or Fisher Scientific (Pittsburgh, Pa.).

A. Cells, Transfection, and Protein Preparation

Cultured cells were maintained in DMEM medium (Life Technologies, Inc., Rockville, Md.) supplemented with 10% fetal bovine serum (Life Technologies, Inc., Rockville, Md.) and 1% antibiotic-antimycotic mixture (Life Technologies, Inc., Rockville, Md.). For transfection, CHO cells were seeded into 6-well plates (Falcon, Franklin Lakes, N.J.), at a concentration of 1.3×10⁴ cells/ml, and maintained in a CO₂ chamber at 37° C. for about 16 h. When 60% confluent, cells were transfected with cDNA of ebaf, using Superfect Transfect Reagent (Qiagen, Valencia, Calif.) or fugene (Boehringer Mannheim, Germany) following the manufacturer's protocol. The amount of proteins in the cell, cytosolic lysates, or nuclear lysates was determined by the Bio-Rad Protein Assay kit (Bio-Rad Laboratories, Hercules, Calif.).

B. Mammalian Expression Plasmid Construction

The ebaf (lefty-A) cDNA was originally cloned in a pBluescript® SK⁻ vector. A forward primer (5′-AGAATTCAAGATGTGGCCCCTGTGGCTCTGCTGGGC-3′) and the reverse primer (5′-TTCTAGACTATGGCTGGAGCCTCCTTGGCACGAGCGCCCC-3′) were used to amplify the coding region of ebaf with the 3′ proofreading polymerase, Pfu (Stratagene, La Jolla, Calif.). The PCR products were separated in 1% agarose gel, and purified with a Geneclean kit (Bio101, LA Jolla, Calif.). The PCR products and the plasmids (pcDNA3 or HA-pcDNA3) were digested with EcoRI and XbaI (New England Lab, Beverly, Mass.). The fragments were annealed to a mammalian expression plasmid (pcDNA3 or HA-pcDNA3) with a Rapid Ligation Kit (Stratagene, Calif.). The sequence of the selected clone was validated by restriction enzyme digestion and by sequencing using Taq DyeDeoxy terminator cycle sequencing reactions in conjunction with an Applied Biosystems model 373 DNA Sequencer. The plasmid DNAs containing the correct cDNA sequence insertions were prepared using the Promega Wizard Miniprep Method (Promega, Madison, Wis.), and used for transfection.

C. Site-Directed Point Mutation of Human Ebaf

The N-glycosylation site of ebaf (amino acid residue 57) was point mutated to “D” using QuikChange™ 1-Day Site-Directed Mutagenesis kit (Stratagene, La Jolla, Calif.), following the manufacturer's protocol. The primers were DRTS-F: 5′-GCGTCCGCGACGACGGCTCCGACCGCACCTCCCTCATCGACTCC-3′; DRTR-R: 5′-GGAGTCGATGAGGGAGGTGCGGTCGGAGCCGTCGTCGCGGACGC-3′. The sequences of all point-mutated clones were determined by Taq DyeDeoxy terminator cycle-sequencing reactions, in conjunction with an Applied Biosystems model 373 DNA Sequencer.

D. Deglycosylation with Endoglycosidase H (Endo H) and Peptide

The concentrated conditioned media and the whole cell lysates of transfected cells were digested for 2-3 h at 37° C. with Endo H and PNGase F (New England Lab, Beverly, Mass.), according to the manufacturer's instructions. The buffer for the digestion consisted of 50 mM sodium citrate (pH 5.5) for Endo H, and 5 mM sodium phosphate (pH 7.5) for PNGase F. The digested proteins then were mixed with Laemmli buffer, denatured by heat, and used for SDS-PAGE and the Western blotting.

E. Affinity-Purification of Ebaf Protein

The inventor made two polyclonal antibodies to ebaf, A351 and A31, by immunizing a rabbit with either the C-terminus of ebaf (amino acid residues 351-367) or the N-terminus of ebaf (amino acid residues 31-43), respectively, and then affinity-purifying the resulting polyclonal antibodies. The inventor then purified ebaf proteins from culture media of HEK-293 cells stably transfected with ebaf. An affinity column was prepared by binding the A351 rabbit anti-ebaf peptide antibody to coupled Sepharose 4B.

The culture media of transfected cells were subjected to Western blotting, followed by staining blot with A351 antibody to verify production of ebaf. Confluent cultures of these cells were incubated overnight with serum-free DMEM medium in a CO₂ incubator. The culture supernatant was collected and centrifuged at 1000×g to remove any cell or cell debris. For purification, urine and the cell-free medium were transferred to fresh tubes, mixed with 200 ml of ebaf-Sepharose 4B suspension, and incubated at room temperature for 1 h with gentle shaking. The Sepharose 4B was separated by centrifugation at 2000×g for 5 min, then washed 3 times in buffer A1 (0.05 M Tris HCl, 0.005 M EDTA, and 0.004 M A ε-aminocaproic acid), followed by 2 washes with buffer A2 (0.05 M Tris HCl, 0.005 M EDTA, 0.005 M ACA, 0.68 M NaCl), pH 7.4. The bound ebaf then was eluted by the addition of 1 ml of buffer B3 (0.06 M Tris HCl, 5 mM EDTA, 10% glycerol, 2% SDS), pH 5.5, at 37° C. for 5 min. The mixture was centrifuged to remove the eluted ebaf. The gel matrix was washed once with 1 ml of elution buffer B3. Supernatant then was added to the first elution buffer. The buffer containing the eluted ebaf then was dialyzed against 3 changes of TE (10 mM Tris HCl and 1 mM EDTA), pH 7.5, at 4° C., and was stored at 4° C.

The amount of protein was measured by a commercially-available protein assay kit (Bio-Rad, Hercules, Calif.). The yield of ebaf was 40 ng for each ml of supernatant of confluent cultures. The two main secreted products of ebaf were separated by gel electrophoresis. The proteins then were eluted from the gel slices. Proteins were rendered visible in the gel by incubation with a buffer containing sodium acetate.

F. SDS-Polyacrylamide Gel Electrophoresis and Western Blotting

Proteins were extracted from tissues by directly placing the tissue in SDS lysis buffer. Ebaf was purified from sera and urine by affinity purification. In some cases, urine was concentrated 250-fold using a Centricon device with the molecular weight cutoff of 10 kD. Proteins were subjected to SDS-PAGE electrophoresis, and transferred to nitrocellulose membranes. Membranes were washed, then incubated with biotin-labeled goat anti-rabbit antiserum (1:2000 dilution) for 90 min at 25° C. The membranes then were washed and incubated with the avidin-biotin-complex (ABC) reagent (1:300 dilution), for 30 min at 25° C., and developed using the enhanced chemiluminescence system. The optical density of each band was determined by laser-scanning densitometry. Kruskal-Wallis and Mann-Whitney tests were used for statistical evaluation. Significance was established at the p<0.05 level.

G. Immunoprecipitation

Immunoprecipitations of the proteins were performed as described (Van de Loo et al., 1997). Briefly, for immunoprecipitation, 2-5 μl of specific antibody was added to each ml of cell lysate (˜10⁶ cells). The immunoprecipitates were subjected to SDS-PAGE and blotting, followed by autoradiography.

3. Results

A. Ebaf Proteins are Secreted.

To permit identification of the various forms of ebaf proteins which are synthesized and released by cells, ebaf cDNA was transfected into several cell lines. A mammalian expression vector containing the coding sequence of ebaf protein was used to transfect human embryonic kidney epithelial (HEK)-293 cells. The secretion of ebaf then was studied using two different assays.

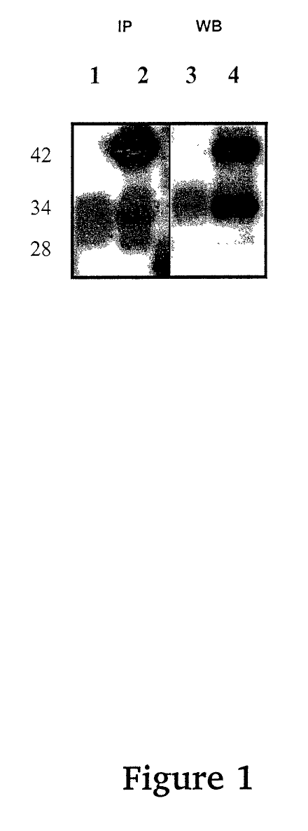

In the first method, proteins were labeled in vivo by ³⁵S-methionine. Ebaf that was secreted into the culture medium was immunoprecipitated with an affinity-purified rabbit polyclonal A351 antibody against a C-terminus peptide of ebaf. The immunoprecipitate was subjected to SDS-PAGE (FIG. 1). In the second method, ebaf in the culture medium of transfected cells was affinity-purified, subjected to Western blotting, then probed using the A351 antibody (FIG. 1). In both assays, ebaf protein was detected as three protein bands of 42-, 34-, and 28-kD proteins in the culture medium of transfected cells. Smaller amounts of the 34- and 28-kD proteins were also found in the culture medium of nontransfected cells showing endogenous production of lower levels of ebaf by these cells. Similar results were obtained when other cell types, including NIH-3T3 cells and CHO cells, were transfected (data not shown).

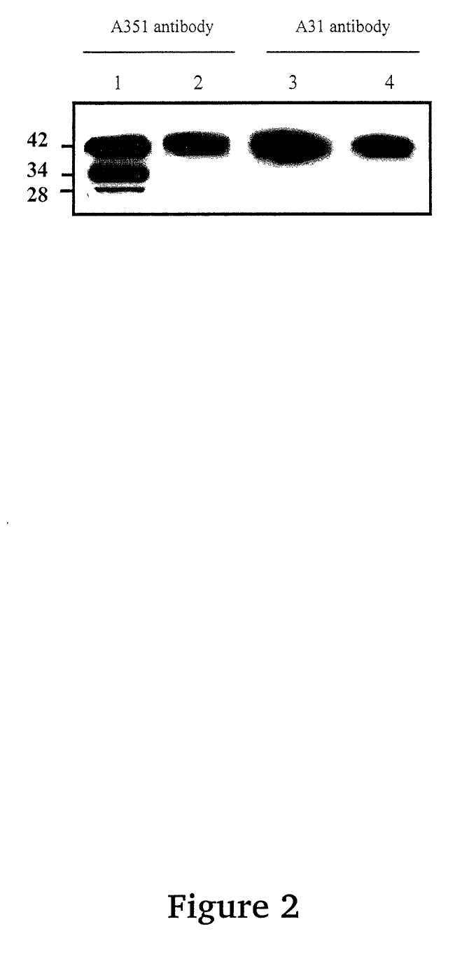

To check the fate of the N-terminus pro-protein region of the ebaf proteins after processing by cells, the inventor generated a polyclonal antibody to an N-terminus peptide. This antibody (A31) was used to analyze the secretion of ebaf by transfected cells. Ebaf was affinity-purified from the culture medium of HEK-293 cells transfected with ebaf cDNA using two affinity columns: one made with the A351 antibody, and the other made with the A31 antibody. The eluates from these columns were subjected to Western blot analysis. One of the two identical blots was stained with the A351 antibody, and the other was stained with the A31 antibody (FIG. 2). Both antibodies detected the 42-kD protein in culture media of transfected cells. However, the 34- and 28-kD proteins only bound to the A351 affinity column; these were detected with the A351 antibody, but not with the A31 antibody. These findings suggest that the 42-kD protein is the precursor polypeptide, and that the 34- and 28-kD proteins are the C-terminus polypeptides processed from the precursor by proteolytic cleavage.

Proteins of the TGF-β superfamily are cleaved by members of the pro-protein convertase (PC) family of endoproteases (Molloy et al., 1992; Dubois et al., 1995; and Cui et al., 1998). These endoproteases are Ca²⁺-dependent serine proteases with a consensus cleavage site of R-X-X-R (Bresnahan et al., 1990; Molloy et al., 1992; Dubois et al., 1995; and Cui et al., 1998). The inventor analyzed the ebaf sequence in order to identify the potential endoproteolytic processing sites of ebaf polypeptides, according to the consensus sequences required for convertase cleavage (Kothapalli et al., 1997). Two sequences, RGKR (amino acid residues 74-77) and RHGR (amino acid residues 132-135), were detected as potential cleavage sites for the processing of ebaf proteins. To ascertain whether these sequences are, indeed, the cleavage sites of ebaf in vivo, the inventor analyzed the effect on ebaf processing of the point mutations RGKR→GGKG (amino acid residues 74-77) and RHGR→GHGR (amino acid residues 132-135).

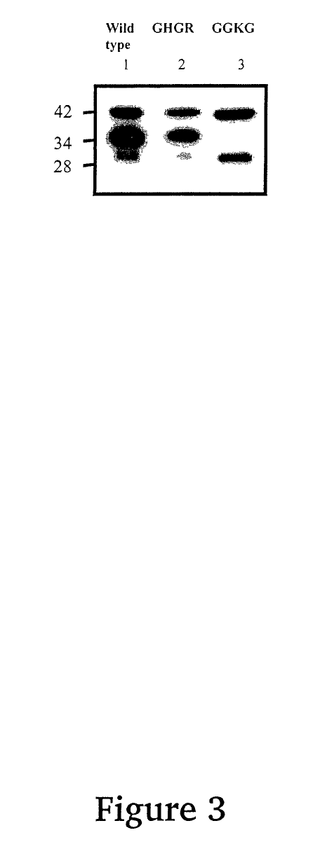

HEK-293 cells were transfected with the cDNA of ebaf mutants, and their culture medium was analyzed by Western blotting (FIG. 3). The mutation RGKR→GGKG (amino acid residues 74-77) inhibited processing of the 42-kD ebaf precursor to the 34-kD form. Contrastingly, the mutation RHGR→GHGR (amino acid residues 132-135) prevented the processing of the 28-kD, but not the 34-kD, polypeptide form. These findings indicate that the proteolytic cleavage of the 28-kD polypeptide occurs independently of the proteolytic cleavage of the 34-kD polypeptide, suggesting that the cleavage at Arg-135 does not require either cleavage at Lys-77 or the formation of the 34-kD polypeptide. These results also rule out the possibility that these point mutations prevented the proteolytic cleavage at other potential cleavage sites.

B. Ebaf Proteins are Glycosylated.

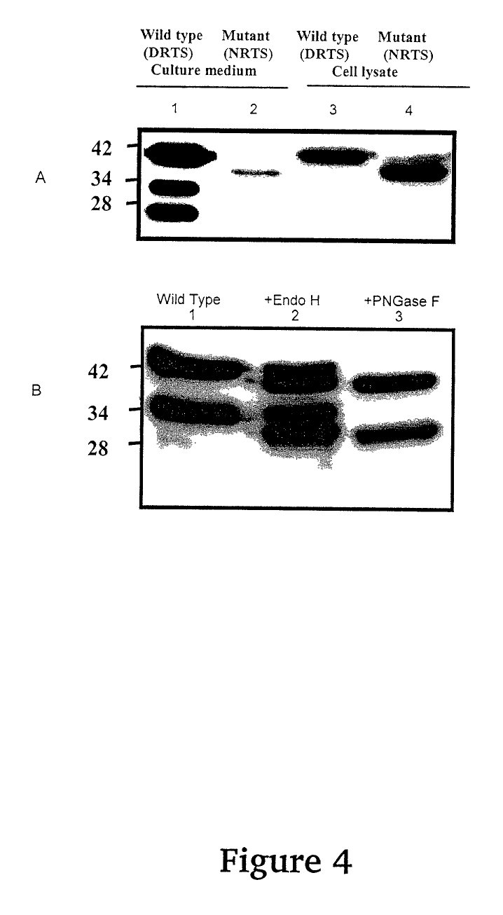

The predicted size of the ebaf protein was smaller than the actual size that was determined for ebaf in immunoprecipitation and Western blot analyses. Moreover, the predicted sizes of the proteins derived from ebaf by cleavage at the RGKR and RHGR sites were also smaller than the actual sizes of these proteins when released into the culture media of transfected cells, suggesting that ebaf undergoes post-translational modification. The inventor detected one potential glycosylation site at amino acid 138 (N) in ebaf protein (Kothapalli et al., 1997). To assess the significance of this site in the glycosylation of ebaf, the inventor mutated “N” to “D”, then transfected HEK-293 cells with the mutant cDNA. Cell lysates and the culture media of transfectants showed an approximately 3-kb reduction in the size of the 42-kD protein (FIG. 4A). Interestingly, the 34- and 28-kD proteins were not detected in the culture media of transfected cells, suggesting that proper processing of ebaf is dependent on glycosylation events.

To directly determine whether ebaf is glycosylated after release into the culture medium, ebaf was digested in vitro with Endo H, an enzyme that cleaves the glycosylated chain of proteins (FIG. 4B) at the high-mannose structures. Endo H digested ebaf to smaller-sized polypeptides. Despite prolonged incubation, digestion with Endo H was incomplete, and both the original-sized proteins and smaller proteins were detected. Therefore, the inventor sought to determine the effect of another enzyme, PNGase F, which is an amidase that cleaves between the innermost GlcNAc and asparagine residues of high-mannose and complex oligosaccharides from N-linked glycoproteins. PNGase F completely digested the 42-kD ebaf, as well as the 34- and 28-kD proteins, and resulted in production of proteins that were about 3-kb smaller in size (FIG. 4B).

C. Ebaf is Synthesized and Released by Bladder Carcinoma.

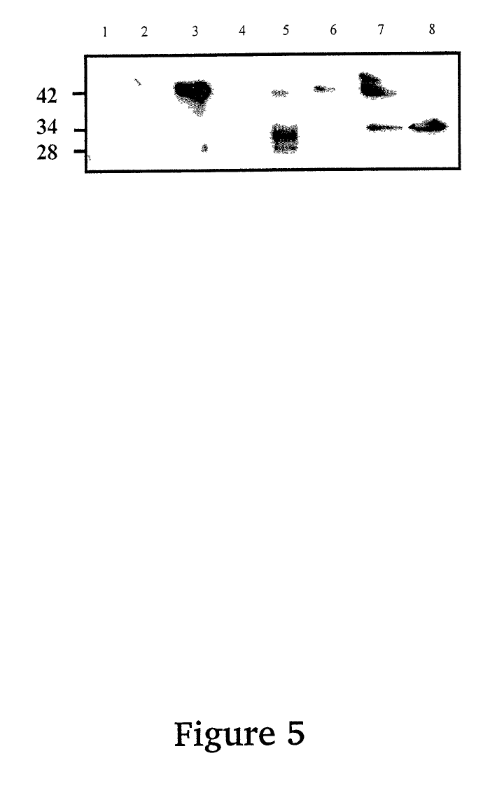

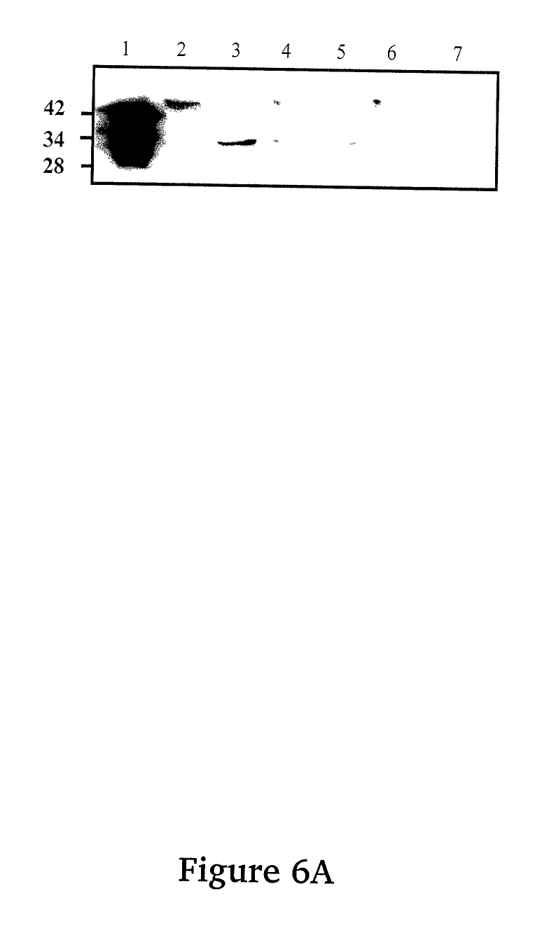

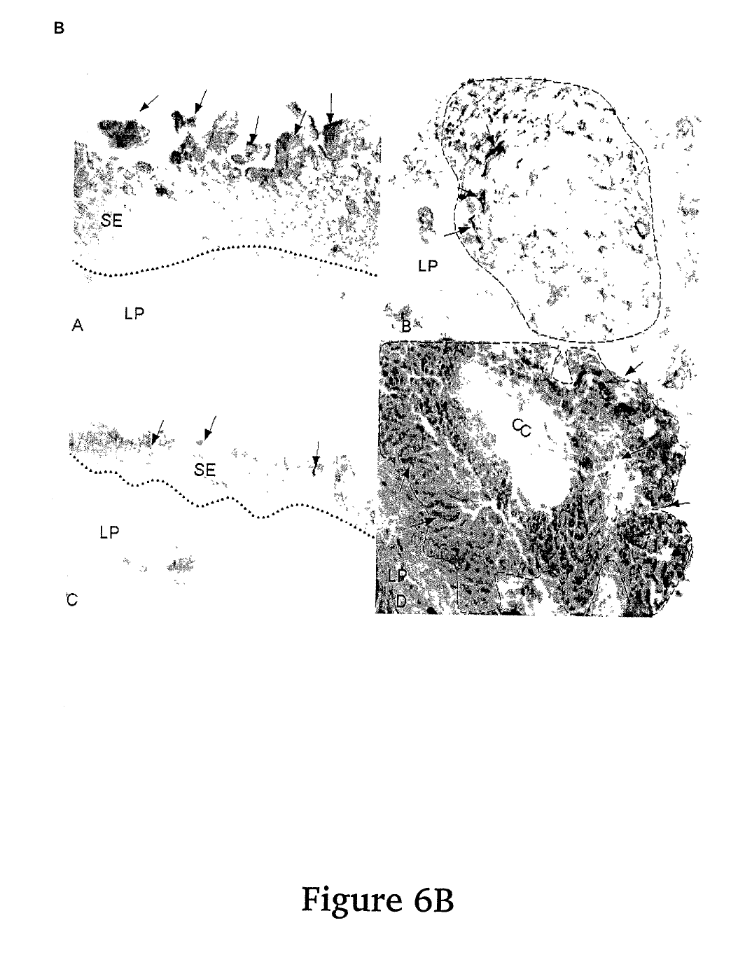

The inventor used bladder carcinoma as a paradigm to test the synthesis and release of ebaf by neoplastic epithelial cells. Tissue lysates of bladder carcinomas were subjected to Western blot analysis using A351 antibody. Ebaf protein was abundant in the bladder carcinoma, as compared with normal bladder mucosa (FIG. 5). The abundance and relative proportion of the 42-, 34-, and 28-kD proteins, however, markedly differed in different carcinomas. In almost all bladder carcinomas, the 42-kD protein was detected. However, the amount of 42- and 28-kD protein varied significantly, implying different aberrations in the processing of ebaf in these cancers. To better validate the relative abundance of ebaf in bladder carcinoma, the amount of ebaf in bladder carcinoma and in adjacent, noninvolved bladder mucosa was assessed by Western blotting. The results of this study showed the 42-kD protein to be more abundant in the carcinoma, as compared with noninvolved mucosa (FIG. 6A). However, the difference in the abundance of processed forms of ebaf in carcinoma, as compared with adjacent bladder mucosa, was less apparent.

To show that ebaf detected in Western blots of bladder cancers is present in the carcinoma cells, rather than in cells in the stroma, tissue sections of the same cases were immunostained. Cryostat sections of the tumors were stained using the A351 anti-ebaf antibody. Ebaf was present both in the atypical epithelium adjacent to the tumors, and in the tumor cells themselves (FIG. 6B).

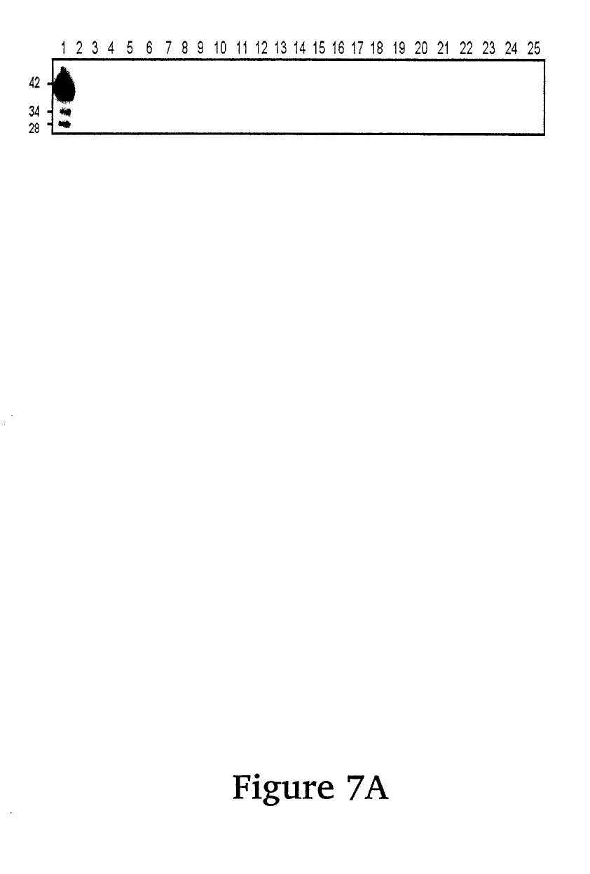

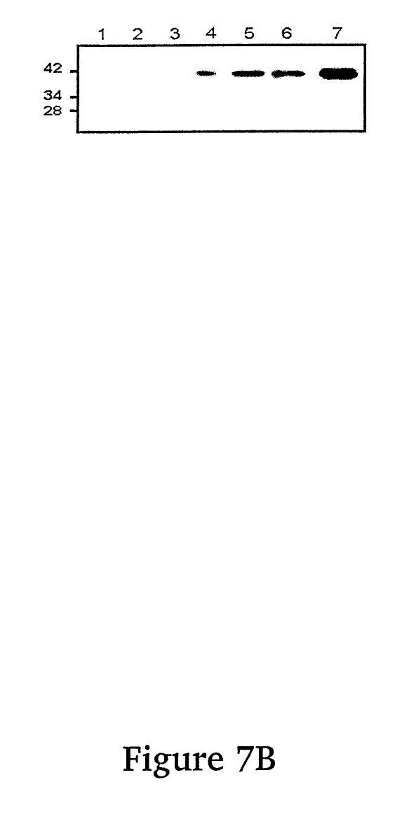

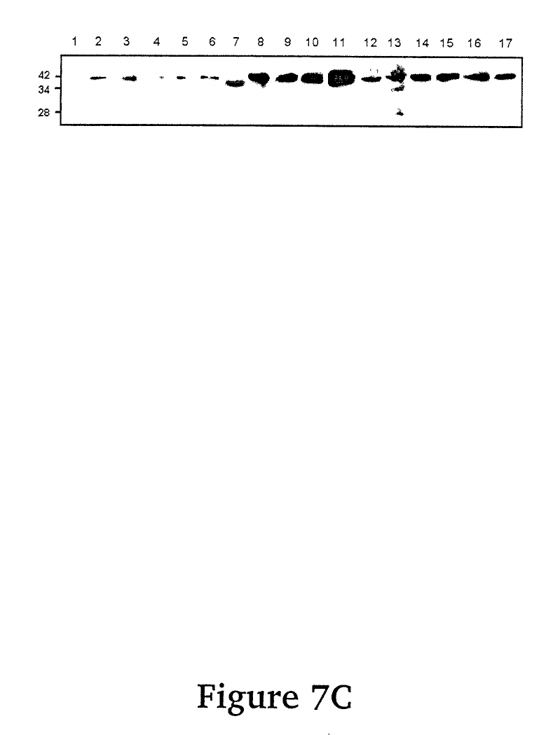

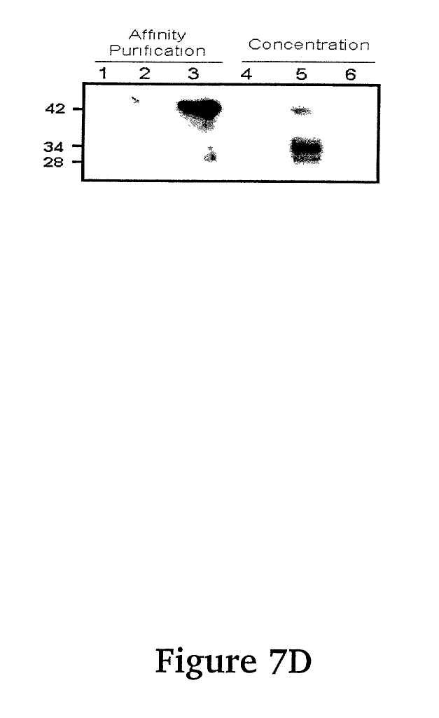

Since ebaf protein has to be secreted to release biologically-active proteins, the amount of ebaf protein in urine also was analyzed. Ebaf protein was affinity-purified from the urine of normal control subjects ranging in age from 7 to 78 years. The purified material then was assessed by Western blot analysis. Very little ebaf was detected in the urine of these subjects (FIG. 7A). Only in three patients were small quantities of 42-kD protein detected. The same analysis then was carried out on the urine of patients with hyperplasia and atypical hyperplasia of urothelium. Significantly higher amounts of ebaf were detected in atypical lesions, as compared with those that showed hyperplasia alone (FIG. 7B). In patients with transitional-cell carcinoma (TCC) of the bladder, significantly higher amounts of ebaf were detected in the urine (FIG. 7C). In most patients with low-grade TCC of the bladder, the amount of ebaf detected in the urine was lower than that detected in patients with high-grade lesions, including carcinoma in situ and high-grade papillary TCC of the bladder (FIG. 7C).

Since the ebaf detected in the urine of bladder cancer patients was mostly the 42-kD form, the amount of ebaf in the urine was directly analyzed in urine samples that had been concentrated using Centricon concentrating device (Amicon Co., Beverly, Mass.) with a 10,000-kD molecular weight cutoff. More of the secreted forms were detectable in the concentrated urine, as compared with those detected by affinity purification of the same amount of urine (FIG. 7D). These findings show a bias of the affinity column, in that it more preferentially allows the elution of the higher molecular-weight protein from the column.

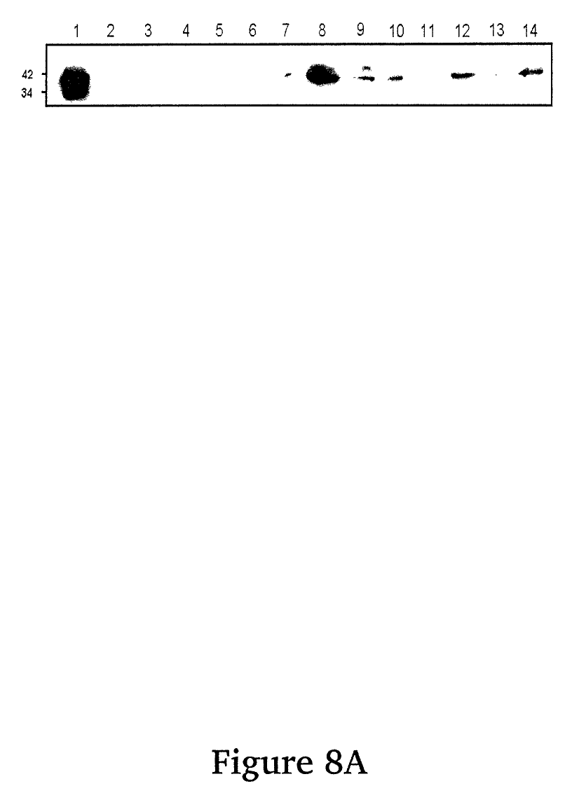

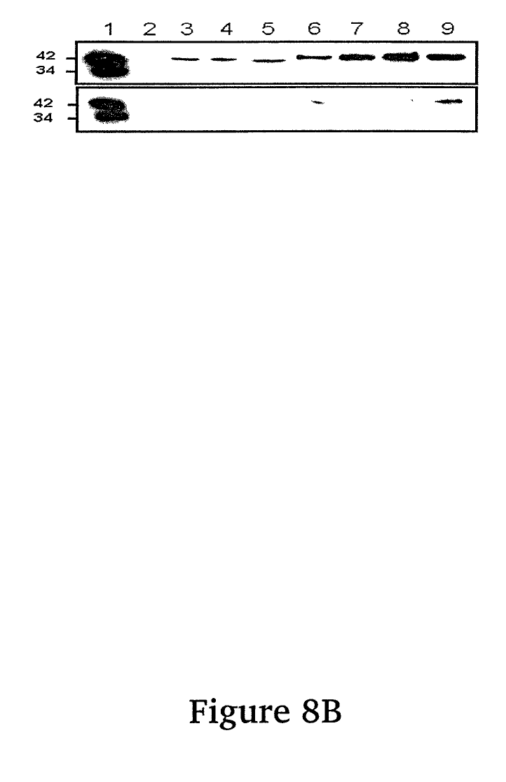

Since ebaf is secreted into the urine of bladder cancer patients, the inventor considered the possibility that ebaf may directly or indirectly gain access to the peripheral circulation. To test this hypothesis, ebaf protein was affinity-purified from the sera of normal male control subjects. Since ebaf is associated with the menstrual cycle, and its expression is enhanced around the time of menses, sera were also obtained from women during the menstrual cycle and around the time of menses. Ebaf was affinity-purified from these sera, and the purified material then was assessed by Western blot analysis. Very little ebaf was detected in the sera of these normal subjects (FIG. 8A). In contrast, significantly higher amounts of ebaf were detected in the sera of patients with bladder carcinomas (FIG. 8A).

To determine whether the amount of ebaf detected in the urine correlated with the amount of ebaf detected in the sera of bladder cancer patients, ebaf was purified from the urine and sera of the same subjects, then subjected to Western blotting. Ebaf was detected both in the urine and sera of these cancer patients. Moreover, more ebaf was found in the sera of patients in whom more ebaf was detected in the urine samples (FIG. 8B). Thus, the amounts appeared to correlate.

4. Discussion

Ebaf protein is overexpressed in certain human carcinomas, but the significance of this overexpression is poorly understood (Tabibzadeh et al., 1997). To better understand the function of this protein, there is a need to identify its processed and biologically-active forms. TGF-β superfamily members are synthesized as large precursors, and their signal peptide is rapidly removed within the cell to produce a large precursor protein. The best-studied example of processing has been shown for TGF-β1, which is expressed as an inactive precursor of 55 kD. TGF-β1 precursor is cleaved to produce the pro-TGF-β1 of 44 kD, and a final polypeptide of 12.5 kD that becomes biologically active in a homodimer form (Dubois et al., 1995).

Herein, the inventor has shown that ebaf is processed to form two cleavage products from a single precursor protein. Transfection with ebaf of HEK-293 and Chinese hamster ovary (CHO) cells showed secretion of three polypeptides of 42, 34, and 28 kD. Characterization of the secreted proteins, both with immunoprecipitation and Western blotting, showed proteins of similar size in the culture media of transfected cells. Smaller quantities of the 34-kD protein were found in the HEK-293 cells, suggesting that ebaf is endogenously synthesized in these cells. In order to identify whether ebaf is endoproteolytically processed, the inventor mutated two potential cleavage sites. The point mutations at the pro-protein convertase (PC) consensus sequences, RGKR (amino acid residues 74-77) and RHGR (amino acid residues 132-135), prevented the formation of the 34-kD and 28-kD ebaf polypeptides, respectively. In view of these data, the inventor suggests that ebaf is synthesized as a 42-kD protein which is proteolytically cleaved at Arg 77 to release the 34-kD polypeptide. The 28-kD ebaf polypeptide is produced by cleavage of the ebaf polypeptide at Arg 132.

The sizes of the proteins released into the culture media of transfected cells were larger than their predicted sizes, suggesting that ebaf undergoes post-translational modifications after synthesis. To show such modification, the inventor mutated the single potential glycosylation site of ebaf. The point mutation, DRTS (amino acid residue 158)→NRTS, led to a loss of approximately 3 kD in the size of the protein, indicating that ebaf is glycosylated at Asp 158. Interestingly, the 34- and 28-kD forms were not detected. Since post-translational modifications, including endoproteolytic processing, take place within the Golgi complex after N-glycosylation (Molloy et al., 1999), the findings imply that a lack of glycosylation events impairs the endoproteolytic processing of ebaf protein. Since point mutation of ebaf did not show whether the 34- and 28-kD proteins are glycosylated, ebaf proteins were digested in vitro with Endo H and PNGase F. Endo H only partially digested ebaf proteins, whereas PNGase F led to complete digestion of ebaf proteins and formation of proteins that were all approximately 3 kD smaller than the undigested forms. These findings confirm that all secreted forms of ebaf proteins are glycosylated.

The N-terminus protein released from proteolytic processing of TGF-β plays a role in the biologic fate of TGF-β. TGF-β is released as a latent complex composed of mature TGF-β1, the N-terminal remnant of the TGF-β1 precursor (TGF-β1-latency associated peptide), and the latent TGF-β1 binding protein (LTBP) (Wakefield et al., 1988; and Miyazono et al., 1991). To address the fate of the N-terminus pro-protein region of the ebaf proteins, the inventor assessed potential presence of the N-terminus protein(s) by Western blotting using the A31 antibody. This analysis showed that the A31 antibody detected the presence of the 42-kD ebaf precursor. However, N-terminus proteins were not detected in the culture medium, thereby suggesting either that they are not secreted or that they are rapidly removed from the culture medium.

The inventor used transitional-cell carcinoma (TCC) of the bladder as a paradigm to analyze the synthesis and secretion of ebaf protein in vivo. Ebaf precursor and its endoproteolytically-processed forms were detected in the bladder carcinoma. The size of the precursor was slightly larger than that detected in the transfected cells, suggesting a more complex post-translational modification in tissues in vivo. Moreover, the extent to which the precursor and processed forms were detected varied among different tumors. In some tumors, the precursor was more abundant; in other tumors, only the 34- and the 28-kD forms were found in excess. This suggests that the proteolytic processing and/or release of ebaf is different or may be impaired in different bladder cancers.

Noninvasive transitional-cell carcinomas of the bladder have two distinct morphologies. Papillary transitional-cell carcinomas are often multifocal, but have a limited potential for invasive growth or a lethal outcome. In contrast, flat tumors, or carcinomas in situ (CIS), frequently progress to invasive cancer (Spruck et al., 1994). In the United States, nearly all cases of bladder cancer are carcinomas of the transitional-cell type. Epidemiological evidence indicates that, among these, approximately 80% initially present as essentially well-differentiated, superficial papillary neoplasms (Farrow, 1992).

Recent studies have revealed genetic alterations in the bladder carcinomas. One of the most common genetic alterations is allelic losses on chromosomes 4, 8, 9, 11, and 17 (Czerniak et al., 2000). The majority of statistically-significant allelic losses (70%) occurred early in low-grade intraurothelial dysplasia, and some of them involved adjacent areas of morphologically-normal mucosa preceding the development of microscopically-recognizable precursor lesions. For this reason, the inventor assessed the amount of ebaf in bladder carcinomas and in the adjacent, grossly-unremarkable tissues. This analysis showed the 42-kD ebaf protein to be more abundant in the bladder carcinomas, while the processed forms of ebaf were more abundant in adjacent tissues. These findings show that progression to carcinoma is associated with either the impairment of synthesis or the release of the processed forms of the ebaf protein.

TGF-β superfamily members are secreted. Therefore, in order to detect the secreted forms of ebaf, the inventor first analyzed urine obtained from normal subjects. There were very small quantities of ebaf in these urine samples. In contrast, the urine of cancer patients contained abundant amounts of ebaf, indicating that ebaf protein is rapidly secreted by the cancer cells into the urine. Although the ebaf protein detected by affinity purification was mostly the 42-kD protein, the processed forms of the proteins were also detected. These processed forms were more frequently observed when ebaf was detected by Western blotting of concentrated urine, rather than by Western blotting of ebaf that had been affinity purified from urine.

The overexpression of ebaf in TCC of the bladder is presumably the consequence of genetic alterations, and is associated with other genetic alterations in bladder carcinomas. Multiple genetic alterations have been described in bladder carcinomas (Orntoft and Wolff, 1998). The loss of heterozygosity has shown a general chromosomal instability in TCC of the bladder, with loss of parts of chromosome 9 in papillomas, and loss of parts of chromosomes 11, 13, 3, 4, 8, 17, and 18 during further development of the tumor. Activation of oncogenes is exemplified by mutations found in the ras gene family, and by overexpression of the c-erbB-2 gene in a smaller number of tumors. The loss of one p53 allele is common in TCC of the bladder, and the expression of p16 and p15, the cell cycle factors, is lost in these tumors. These alterations correlate well with survival, disease progression, invasion, and recurrence of TCC of the bladder. The downregulation of the ABO gene, followed by loss of ABO blood group structures and accumulation of the Lewis cell adhesion molecules, has been reported in high-grade tumors (Orntoft and Wolff, 1998).

Similar to the findings reported here, elevated levels of TGF-β1 was found in the sera of patients with invasive bladder carcinoma (Eder et al., 1996). Serum TGF-β1 levels were significantly elevated in patients with invasive bladder cancer (61.5 ng/ml), as compared with 18 healthy controls (36.3 ng/ml). Moreover, the expression of TGF-β1 protein was detected at higher levels in superficial bladder tumors (mean level of 0.153 ng/mg protein) and in invasive T2/T3 tumors (mean level of 0.104 ng/mg protein), as compared with normal urothelium (mean level of 0.065 ng/mg protein) (Eder et al., 1997). In a separate study, there were significantly higher levels of TGF-β1 transcripts in low- and intermediate-grade (Grade 1 and 2) tumors than in high-grade (Grade 3) tumors (p<0.02). Superficial (pTa and pT1) tumors had higher levels of TGF-β1 than did invasive (pT2 or higher) tumors (p<0.05) (Miyamoto et al., 1995).

Recently, other biomarkers that are correlated with grade and stage of TCC of the bladder have been identified by functional proteome analysis (Orntoft and Wolff, 1998). Biomarkers currently used for screening and detection of bladder cancer are the nuclear matrix protein, NMP-22; human complement factor H related protein (BTA stat); FDP; chemiluminescent hemoglobin; hemoglobin dipstick; and telomerase assay. Among these, telomerase assay seems to offer greater sensitivity and specificity (Landman et al., 1998; and Ramakumar et al., 1999). Quantification of ebaf also may be a useful biomarker for screening for TCC of the bladder, and potentially may be used for determining and predicting the survival, disease progression, invasion, and recurrence of the disease by analysis of urine, serum, other bodily fluids, and cells or tissues obtained from the urinary tract.

The spectrum of changes detected in bladder have been recently re-classified by the World Health Organization. According to the new classification, flat urothelial lesions include hyperplasia, reactive atypia/atypia of unknown significance, dysplasia, and carcinoma in situ. Papillary urothelial lesions include papillomas, papillary neoplasms of low malignant potential, and papillary carcinomas (Helpap et al., 2000). Separation of the bladder tumors used in this study, in accordance with this classification, revealed that ebaf was detected at a low level in the urine of patients with hyperplasia, but that significantly higher levels were found in the urine of patients with flat carcinoma in situ. In the majority of patients who had low-grade papillary transitional-cell carcinoma, amounts of ebaf found in the urine were lower than the amounts of ebaf found in the urine of patients with high-grade carcinomas. These findings show that the overexpression of ebaf correlates with the degree of morphologic differentiation of transitional-cell carcinomas, with those having a poor prognosis showing the potential to secrete more ebaf into the urine.

### REFERENCES

1. Baniel, J., Bladder cancer in women. Int. Urogynecol. J. Pelvic Floor Dysfunct., 10(6):399-404, 1999.
2. Beers and Berkow, eds., The Merck Manual of Diagnosis and Therapy, 17^{th} ed. (Whitehouse Station, N.J.: Merck Research Laboratories, 1999) 1814, 1917.
3. Bresnahan et al., Human fur gene encodes a yeast KEX2-like endoprotease that cleaves pro-beta-NGF in vivo. J. Cell Biol., 111:2851-59, 1990.
4. Cui et al., BMP-4 is proteolytically activated by furin and/or PC6 during vertebrate embryonic development. EMBO J., 17(16):4735-43, 1998.
5. Czerniak et al., Genetic modeling of human urinary bladder carcinogenesis. Genes Chromosomes Cancer, 27(4):392-402, 2000.
6. Dubois et al., Processing of transforming growth factor beta 1 precursor by human furin convertase. J. Biol. Chem., 270(18):10618-24, 1995.
7. Eder et al., Transforming growth factors-beta 1 and beta 2 in serum and urine from patients with bladder carcinoma. J. Urol., 156(3):953-57, 961, 1996.
8. Eder et al., Expression of transforming growth factors beta-1, beta 2 and beta 3 in human bladder carcinomas. Br. J. Cancer, 75(12):1753-60, 1997.
9. Farrow, G. M., Pathology of carcinoma in situ of the urinary bladder and related lesions. J. Cell. Biochem., Suppl; 161:39-43, 1992.
10. Helpap and Kollermann, Revisions in the WHO histological classification of urothelial bladder tumors and flat urothelial lesions. 21(3):211-17, 2000.
11. Kosaki et al., Characterization and mutation analysis of human LEFTY A and LEFTY B, homologues of murine genes implicated in left-right axis development, Am. J. Hum. Genet, 64(3):712-21, March 1999.
12. Kothapalli et al., Detection of ebaf, a novel human gene of the TGF-β superfamily; association of gene expression with endometrial bleeding J. Clin. Invest., 99:2342-50, 1997.
13. Lamb et al., Neural induction by the secreted polypeptide noggin. Science, 262(5134):713-18, 1993.
14. Landman et al., Sensitivity and specificity of NMP-22, telomerase, and BTA in the detection of human bladder cancer. Urology, 52(3):398-402, 1998.
15. Madrid Garcia et al., Transitional cell bladder carcinoma in patients younger than 40 years of age. Arch. Esp. Urol., 51(10):991-4, 1998.
16. Meno et al., Left-right asymmetric expression of the TGF beta-family member lefty in mouse embryos. Nature, 381(6578):151-5, 1996.
18. Meno et al., Two closely-related left-right asymmetrically expressed genes, lefty-1 and lefty-2: their distinct expression domains, chromosomal linkage and direct neuralizing activity in Xenopus embryos. Genes Cells, 2(8):513-24, 1997.
19. Meno et al., Lefty-1 is required for left-right determination as a regulator of lefty-2 and nodal. Cell, 94(3):287-97, 1998.
20. Metts et al., Bladder cancer: a review of diagnosis and management. J. Natl. Med. Assoc., 92(6):285-94, 2000.
21. Miyamoto et al., Expression of transforming growth factor-beta 1 in human bladder cancer. Cancer, 75(10):2565-70, 1995.
22. Miyazono et al., A role of the latent TGF-beta 1-binding protein in the assembly and secretion of TGF-beta 1. EMBO J., 10(5):1091-101, 1991.
23. Molloy et al., Human furin is a calcium-dependent serine endoprotease that recognizes the sequence Arg-X-X-Arg and efficiently cleaves anthrax toxin protective antigen. J. Biol. Chem., 267(23):16396-402, 1992.
24. Molloy et al., Bi-cycling the furin pathway: from TGN localization to pathogen activation and embryogenesis. Trends Cell Biol., 9(1):28-35, 1999.
25. Orntoft and Wolff, Molecular alterations in bladder cancer. Urol. Res., 26(4):223-33, 1998.
26. Ramakumar et al., Comparison of screening methods in the detection of bladder cancer. J. Urol., 161(2):388-94, 1999.
27. Seth et al., Vary CP Coordinate expression of novel genes during osteoblast differentiation. J. Bone Miner. Res., 15(9):1683-96, 2000.
28. Sha et al., Transforming growth factor beta 1: importance of glycosylation and acidic proteases for processing and secretion. Mol. Endocrinol, 3:1090-98, 1989.
29. Soloway, M. S., Managing superficial bladder cancer: an overview. Urology, 40(6 Suppl.):5-10, 1992.
30. Spruck et al., Two molecular pathways to transitional cell carcinoma of the bladder. Cancer Res., 54(3):784-88, 1994.
32. Tabibzadeh et al., Distinct tumor specific expression of TGFB4 (ebaf), a novel human gene of the TGF-beta superfamily. Front. Biosci., 2:a18-25, 1997.
33. Van de Loo et al., Biosynthesis, distinct post-translational modifications, and functional characterization of lymphoma proprotein convertase. J. Biol. Chem., 272(43):27116-23, 1997.
34. Wakefield et al., Latent transforming growth factor-beta from human platelets. A high molecular weight complex containing precursor sequences. J. Biol. Chem., 263(16):7646-54, 1988.

All publications mentioned hereinabove are hereby incorporated in their entireties. While the foregoing invention has been described in some detail for purposes of clarity and understanding, it will be appreciated by one skilled in the art, from a reading of the disclosure, that various changes in form and detail can be made without departing from the true scope of the invention in the appended claims.

What is claimed is:

1. A method for determining whether a subject has a pre-neoplastic or neoplastic lesion in transitional epithelial cells, comprising assaying a diagnostic sample of the subject for ebaf expression, wherein ebaf expression elevated above normal is diagnostic of a pre-neoplastic or neoplastic lesion in transitional epithelial cells.

2. The method of claim 1, wherein the pre-neoplastic lesion in transitional epithelial cells is urothelial atypia/dysplasia.

3. The method of claim 1, wherein the neoplastic lesion in transitional epithelial cells is a transitional-cell carcinoma of the urinary tract.

4. The method of claim 1, wherein the diagnostic sample is tissue, bodily fluid, or a cytological preparation.

5. The method of claim 4, wherein the tissue is selected from the group consisting of bladder tissue, tissue of the pelvis of the kidney, ureter tissue, or urethral tissue.

6. The method of claim 4, wherein the bodily fluid is selected from the group consisting of cerebrospinal fluid, pericardial fluid, peritoneal fluid, saliva, serum, and urine.

7. The method of claim 1, wherein the diagnostic sample is assayed using an agent reactive with ebaf.

8. The method of claim 7, wherein the agent is labeled with a detectable marker.

9. The method of claim 7, wherein the agent is an antibody.

10. The method of claim 9, wherein the antibody is labeled with a detectable marker.

11. The method of claim 1, wherein the diagnostic sample is assayed using at least one nucleic acid probe which hybridizes to nucleic acid encoding ebaf.

12. The method of claim 11, wherein the nucleic acid probe is DNA or RNA.

13. The method of claim 12, wherein the nucleic acid probe is labeled with a detectable marker.

14. A method for assessing the efficacy of therapy to treat a pre-neoplastic or neoplastic lesion in transitional epithelial cells in a subject who has undergone or is undergoing treatment for a pre-neoplastic or neoplastic lesion in transitional epithelial cells, comprising assaying a diagnostic sample of the subject for ebaf expression, wherein normal ebaf expression is indicative of successful therapy to treat a pre-neoplastic or neoplastic lesion in transitional epithelial cells, and ebaf expression elevated above normal is indicative of a need to continue therapy to treat a pre-neoplastic or neoplastic lesion in transitional epithelial cells.

15. The method of claim 14, wherein the pre-neoplastic lesion in transitional epithelial cells is urothelial atypia/dysplasia.

16. The method of claim 14, wherein the neoplastic lesion in transitional epithelial cells is a transitional-cell carcinoma of the urinary tract.

17. The method of claim 14, wherein the diagnostic sample is assayed using an agent reactive with ebaf.

18. The method of claim 17, wherein the agent is labeled with a detectable marker.

19. The method of claim 17, wherein the agent is an antibody.

20. The method of claim 19, wherein the antibody is labeled with a detectable marker.

21. The method of claim 14, wherein the diagnostic sample is assayed using at least one nucleic acid probe which hybridizes to nucleic acid encoding ebaf.

22. The method of claim 21, wherein the nucleic acid probe is DNA or RNA.

23. The method of claim 22, wherein the nucleic acid probe is labeled with a detectable marker.

24. A method for assessing the prognosis of a subject who has a pre-neoplastic or neoplastic lesion in transitional epithelial cells, comprising assaying a diagnostic sample of the subject for ebaf expression, wherein the subject's prognosis improves with a decrease in ebaf expression in the diagnostic sample of the subject.

25. The method of claim 24, wherein the pre-neoplastic lesion in transitional epithelial cells is urothelial atypia/dysplasia.

26. The method of claim 24, wherein the neoplastic lesion in transitional epithelial cells is a transitional-cell carcinoma of the urinary tract.
